(19) United States
(12) Patent Application Publication
(10) Pub. No.: US 2018/0074080 A1
(43) Pub. Date: Mar. 15, 2018

(54) NANOPARTICLES AS CATALYTIC SUBSTRATES FOR REAL-TIME BIOSENSING OF HUMAN PERFORMANCE AND DIAGNOSTIC AND THERAPEUTIC METHODS

(71) Applicant: Northwestern University, Evanston, IL (US)

(72) Inventors: C. Shad Thaxton, Chicago, IL (US); Rohun U. Palekar, Chicago, IL (US); Kaylin M. McMahon, Chicago, IL (US)

(21) Appl. No.: 15/706,648

(22) Filed: Sep. 15, 2017

Related U.S. Application Data
(60) Provisional application No. 62/395,245, filed on Sep. 15, 2016.

(51) Int. Cl. G01N 33/92; G01N 33/52; H01F 1/00

(52) U.S. Cl. CPC G01N 33/92; B82Y 30/00; H01F 1/0054; G01N 33/52

(57) ABSTRACT

Nanostructures having an inorganic core and a lipid layer capable of binding a lecithin:cholesterol acyltransferase (LCAT) activator such as an apolipoprotein are provided herein. Methods of using the nanostructures and related devices and compositions for assessing the risk of developing a disease or condition or treating the disease or condition are also provided.

### RELATED APPLICATIONS

This application claims the benefit under 35 U.S.C. § 119(e) of U.S. provisional application Ser. No. 62/395,245, filed Sep. 15, 2016, the entire contents of which is incorporated by reference herein.

### GOVERNMENT SUPPORT

This invention was made with government support under FA8650-15-2-5518 awarded by the Air Force Materiel Command Legal Office (AFMCLO/JAZ). The government has certain rights in the invention.

### FIELD OF INVENTION

The present invention generally relates to detection of metabolic markers and enzymatic markers and related products and methods.

### BACKGROUND

Although several tests measure indirect aspects of exercise, there are currently no existing tests that allow for precise and sensitive tracking of the physiological effects of exercise in real-time.

### SUMMARY OF THE INVENTION

The invention in some aspects is a device comprising: a housing, a blood extraction element connected at least in part, either directly or indirectly to an external portion of the housing, a nanostructure capable of binding an LCAT activator within at least a portion of the housing. In some embodiments the device is a wearable or portable device.

In other embodiments the nanostructure comprises a solid core and a lipid layer.

The LCAT activator is an apolipoprotein in some embodiments.

The nanostructure has a gold core and a lipid bilayer or monolayer in other embodiments.

In some aspects the invention is a method for rapid detection of an exercise or metabolic associated enzyme comprising: contacting a biological sample with a labeled nanoparticle containing lipids capable of binding an LCAT activator, incubating the nanoparticle with the biological sample for at least 15 minutes, measuring LCAT activation as an indicator of the presence of the exercise associated enzyme in the biological sample. In some embodiments the biological sample is blood.

In some embodiments the LCAT activator is an apolipoprotein.

In other embodiments the label is a fluorescent label. In yet other embodiments the fluorescent label is on a phospholipid in the nanostructure.

The method is performed in vitro in some embodiments. In some embodiments the biological sample is isolated from the subject and the method is performed by using a wearable or portable device.

In some embodiments the nanostructure comprises a nanostructure core comprising an inorganic material, a shell comprising a lipid layer surrounding and attached to the nanostructure core, the shell having a phospholipid monolayer or bilayer having an inner surface and an outer surface.

In some embodiments at least 80% of the lipids in the shell are natural phospholipids. In yet other embodiments the lipids in the shell are comprised of 1,2-dipalmitoyl-sn-glycero-3-phosphothioethanol (DPPTE), phosphotidylcholine (PC) and 1,2-dipalmitoyl-sn-glycero-3-phosphocholine (DPPC).

The invention in other aspects is a composition comprising a fluorescently labeled lipid such as phophatidyl choline or cholesterol lipid.

Optionally the structure includes an apolipoprotein. The lipid shell may be a lipid bilayer or a lipid monolayer.

In some embodiments the core is an inorganic core such as a gold core. In embodiments the structure has 60-250 fold excess lipid to gold core. In other embodiments the core is an organic core.

According to another aspect, the invention is a method assay for measuring HDL function, wherein the method includes contacting a solution of nanoparticles that are comprised of a nanostructure core comprising an inorganic material, a lipid layer, surrounding and attached to the nanostructure core, the shell having a monolayer or bilayer of lipids with a solution that has an apolipoportein, wherein in some cases the apolipoportein is apolipoportein A-I, and, in some cases, the solution also contains lecithin:cholesterol acyl transferase.

According to another aspect, the invention is a method for determining the risk for developing a cardiovascular disease or condition in a subject, the method comprising obtaining a biological sample from the subject, contacting the biological sample with a nanostructure, wherein the nanostructure comprises a nanostructure core comprising an inorganic material; a lipid layer, surrounding and attached to the nanostructure core, the shell having an inner surface and an outer surface, incubating the nanostructure with the biological sample for a time sufficient to sequester one or more apolipoproteins and, in some cases, cholesterol from the sample, detecting the amount of cholesteryl ester formed, comparing the amount of cholesteryl ester formed in the biological sample with a predetermined value, wherein the predetermined value represents the level of cholesteryl ester formed in a subject with the potential for having reduced risk of a cardiovascular disease or condition, and determining that the subject is at reduced risk of developing the cardiovascular disease or condition if the amount of cholesteryl ester formed in the biological sample is at or above the predetermined value or that the subject is at increased risk of developing the cardiovascular disease or condition if the amount of cholesteryl ester formed in the biological sample is below the predetermined value.

According to another aspect, the invention is a method for assessing the effect of one or more interventions on improving a cardiovascular disease or condition in a subject, the method comprising obtaining a biological sample from the subject, contacting the biological sample with a nanostructure, wherein the nanostructure comprises a nanostructure core comprising an inorganic material, a shell comprising a lipid layer, surrounding and attached to the nanostructure core, the shell having an inner surface and an outer surface, incubating the nanostructure with the sample for a time sufficient to sequester one or more apolipoprotein and, in some cases, cholesterol molecules from the biological sample, detecting the level of cholesteryl ester formed, exposing the subject to one or more interventions and obtaining a biological sample from the subject, contacting the biological sample with a nanostructure, wherein the nanostructure comprises a nanostructure core comprising an inorganic material, a shell comprising a lipid layer, surrounding and attached to the nanostructure core, the shell having an inner surface and an outer surface, incubating the nanostructure with the sample for a time sufficient to sequester one or more apolipoprotein and, in some cases cholesterol, from the biological sample, detecting the level of cholesteryl ester formed, comparing the levels of cholesteryl ester formed before the one or more interventions with the levels of cholesteryl ester formed after the one or more interventions, and determining that the one or more interventions improved the cardiovascular disease risk or condition if the amount of cholesteryl ester formed after the one or more interventions is above the level cholesteryl ester formed before the one or more interventions or that the one or more interventions did not improve the cardiovascular disease risk or condition if the level of cholesteryl ester formed after the one or more interventions is at or below the level of cholesteryl ester formed before the one or more interventions.

In some embodiments, the lipid layer is a lipid bilayer or monolayer.

In some embodiments, at least 80%, 85%, 90%, 95%, or 98% of the lipids in the shell are natural phospholipids. In some embodiments, the lipids in the shell are comprised of 1,2-dipalmitoyl-sn-glycero-3-phosphothioethanol (DPPTE), phosphotidylcholine (PC) and 1,2-dipalmitoyl-sn-glycero-3-phosphocholine (DPPC).

In some embodiments, the core is a gold core. In some embodiments, the gold core is 5-6 nm in diameter.

In some embodiments, the apolipoprotein is apolipoprotein AI (Apo-AI). In some embodiments, there are 2-4 apolipoprotein-AI molecules on the nanostructure.

In some embodiments, there are 71-95 lipids in the outer surface of the shell. In some embodiments, the lipids are phospholipids.

In some embodiments, the nanostructure is incubated with the biological sample for about one hour.

In some embodiments, the solution is a phosphate buffered saline (PBS) solution. In some embodiments, the solution is serum. In some embodiments, the serum is diluted to a concentration of 0.1%, 0.5%, 1%, 10%, or is not diluted, at all.

In some embodiments, the serum is depleted from ApoB. In certain embodiments, the serum is depleted from ApoB using PEG8000.

In some embodiments, the subject is a mammal. In some embodiments, the subject is a human.

In any of the embodiments described herein involving the measurement of LCAT activity, the measurement of LCAT activity may be replaced with an alternative or additional measurement of bound protein, as well as the enzymatic activity. In some instances the bound protein is ApoAI (also referred to as a specific activator of an enzyme (LCAT)) The amount of cholesteryl ester detected correlates with the amount of apoAI sequestered by the particles and both may be detected alternatively or additionally. The lipid functionalized nanoparticles may be added to a solution whereby it specifically sequesters a protein associated with metabolism or that is important for cardiovascular disease risk assessment (e.g. apoAI, apoB100, etc.) and then the amount of protein bound is measured by measuring the amount of gold nanoparticle with the protein bound. This could be achieved through a binding assay such as an antibody binding assay that captures apoA1 on and off the particles, but one uses the gold as a way to see exactly how much has been bound by gold (e.g. through a colorimetric, silver enhancement, electric, spectroscopic measurement, etc.).

In some embodiments, the method further comprises isolating the nanoparticles to measure the levels of the metabolic associated protein that binds to the surface of the lipid functionalized nanoparticle. In some embodiments the protein is apolipoprotein A-I. The protein is apolipoportein B100 in other embodiments. The adsorbed protein could be detected by colorimetric, spectroscopic, electrical, or by enhancement techniques known to those skilled in the art based upon the presence of the inorganic nanoparticle core. The core could be a gold nanoparticle. The core could be a magnetic nanoparticle in some embodiments.

In some embodiments, the method further comprises isolating the nanoparticles to measure the levels of cholesteryl ester.

In other embodiments, the method further comprises isolating the nanoparticles to measure the levels of LCAT activator bound to the nanoparticles.

In some embodiments, the cholesteryl ester is measured through a colorimetric assay and wherein the levels of cholesteryl ester directly correlate with apoAI in the biological sample.

In some embodiments, the intervention is a therapeutic intervention. In some embodiments, the intervention is exercise. In some embodiments, the intervention is a dietary modification.

In some embodiments, the biological sample is serum. In some embodiments, the serum is diluted to a concentration of 0.1%, 0.5%, 1% or 10%, or not at all. In some embodiments, the serum is diluted to a concentration of 1%.

In some embodiments, the nanostructure further comprises LCAT. In some embodiments, the nanostructure further comprises one or more cholesterol molecules.

According to another aspect, the invention is a method for synthesizing a nanostructure in situ, the method comprising incubating a nanostructure comprising a nanostructure core comprising an inorganic material, a lipid layer, surrounding and attached to the nanostructure core, the shell having an inner surface and an outer surface, with a biological sample for a time sufficient to sequester one or more apolipoproteins and, in some cases cholesterol, from the biological sample.

In some embodiments, the lipid layer is a lipid bilayer.

In some embodiments, the lipids in the shell are comprised of 1,2-dipalmitoyl-sn-glycero-3-phosphothioethanol (DPPTE), phosphotidylcholine (PC) and 1,2-dipalmitoyl-sn-glycero-3-phosphocholine (DPPC).

In some embodiments, the core is a gold core. In some embodiments, the gold core is 5-6 nm in diameter.

In some embodiments, the apolipoprotein is apolipoprotein AI (Apo-AI). In some embodiments, there are 2-4 apolipoprotein-AI molecules on the nanostructure.

In some embodiments, there are 71-95 lipids in the outer surface of the shell. In some embodiments, the lipids are phospholipids.

In some embodiments, the biological sample is serum. In some embodiments, the serum is diluted to a concentration of 0.1%, 0.5%, 1% or 10%. In some embodiments, the serum is diluted to a concentration of 1%.

In some embodiments, the nanostructure further comprises LCAT. In some embodiments, the nanostructure further comprises one or more cholesterol molecules.

According to another aspect, the invention is a kit for measuring high density lipoprotein (HDL) function, the kit comprising a nanostructure comprising a nanostructure core comprising an inorganic material, a shell comprising a lipid layer, surrounding and attached to the nanostructure core, the shell having an inner surface to be incubated with a biological sample for a time sufficient to sequester one or more apolipoproteins and, in some cases, cholesterol from the biological sample.

In some embodiments, the lipid layer is a lipid bilayer.

In some embodiments, the lipids in the shell are comprised of 1,2-dipalmitoyl-sn-glycero-3-phosphothioethanol (DPPTE), phosphotidylcholine (PC) and 1,2-dipalmitoyl-sn-glycero-3-phosphocholine (DPPC).

In some embodiments, the core is a gold core. In some embodiments, the gold core is 5-6 nm in diameter.

In some embodiments, the apolipoprotein is apolipoprotein AI (Apo-AI). In some embodiments, there are 2-4 apolipoprotein-AI molecules on the nanostructure.

In some embodiments, there are 71-95 lipids in the outer surface of the shell. In some embodiments, the lipids are phospholipids.

In some embodiments, the biological sample is serum. In some embodiments, the serum is diluted to a concentration of 0.1%, 0.5%, 1% or 10%.

In some embodiments, the nanostructure further comprises LCAT. In some embodiments, the nanostructure further comprises one or more cholesterol molecules.

According to another aspect, the invention is a method for synthesizing a nanostructure in situ, the method comprising incubating a nanostructure comprising a nanostructure inorganic core, a lipid shell, surrounding and attached to the nanostructure inorganic core, the shell having an inner surface and/or an outer surface, with a biological sample for a time sufficient to sequester one or more apolipoproteins and, in some cases cholesterol, from the biological sample. The in situ formed nanostructure is then used as a therapeutic either after administering the nanostructure or upon administering the biological sample now containing the in situ formed nanostructures to an individual.

In other aspects, the invention is a method for synthesizing a nanostructure in situ, the method comprising incubating a nanostructure comprising an inorganic core, a lipid shell, surrounding and attached to the inorganic core, the shell having an inner surface and/or an outer surface, with a biological sample for a time sufficient to sequester one or more apolipoproteins present in the biological sample.

In some embodiments the nanostructure sequesters cholesterol. In other embodiments the method further involves administering the biological sample to a subject as a therapeutic.

In other aspects the invention is a method for sequestering cholesterol in a subject by administering to a subject a nanostructure consisting essentially of an inorganic core, a lipid shell, surrounding and attached to the inorganic core, the shell having an inner surface and/or an outer surface, wherein the nanostructure is capable of sequestering apolipoprotein in vivo, which sequesters cholesterol.

In some embodiments the lipid shell is comprised of phospholipids. In other embodiments the subject has a disease associated with high cholesterol. The disease associated with high cholesterol may be selected from the group consisting of cardiovascular disease, atherosclerosis, hyperlipidemia, cancer, inflammation, a protein storage disease, a disease of hemostasis, a rheumatic disease, or a neurologic disease.

A therapeutic or diagnostic composition is provided in other aspects of the invention. The composition is a nanostructure consisting essentially of an inorganic core and a lipid shell, surrounding and attached to the inorganic core, wherein the nanoparticle is formulated in a pharmaceutically acceptable carrier. The composition may be a prodrug.

In some embodiments the lipid shell is a lipid bilayer or a lipid monolayer. In other embodiments the lipids in the shell are comprised of 1,2-dipalmitoyl-sn-glycero-3-phosphothioethanol (DPPTE), phosphotidylcholine (PC) and 1,2-dipalmitoyl-sn-glycero-3-phosphocholine (DPPC).

In some embodiments the core is a gold core that is optionally 5-6 nm in diameter.

In some embodiments the apolipoprotein is apolipoprotein AI (Apo-AI). The nanostructure may be constructed and arranged to sequester 2-4 Apo-AI molecules and to optionally have 71-95 lipids in the shell.

In some embodiments the lipids are phospholipids.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. The details of one or more embodiments of the invention are set forth in the accompanying Detailed Description, Examples, Claims, and Figures Other features, objects, and advantages of the invention will be apparent from the description and from the claims.

The terms nanoparticles and nanostructures are used interchangeably herein.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 shows a schematic depicting the formation of HDL-NP with each component step, with TopFluor PC used as an example fluorescent lipid.

FIG. 2 is a set of graphs depicting the results of an assay incorporating a fluorescent phosphotidylcholine into the outer leaflet of the HDL-NP to demonstrate that incubation of fluorescent HDL-NP with mixtures of purified LCAT and cholesterol for 24 hours results in an increase in fluorescence using both TopFluor labeled PC (FIG. 2, left) and TopFluor TMR labeled PC (FIG. 2, right).

FIG. 3 is a graph depicting the results of an assay utilizing a variant of the TopFluor-loaded HDL-NP without ApoAI, termed the Bilayer NP. The specificity of the HDL-NP for LCAT activity is shown in the data of FIG. 3, which demonstrates that in the presence of cholesterol and LCAT, the fluorescent signal from TopFluor HDL NP is significantly increased over the Bilayer NP group (p=0.00012), and that Bilayer NPs loaded with cholesterol show no significant change in fluorescent signal with or without LCAT (p=0.22).

FIG. 4 is a graph depicting the results of an assay showing that through incubations of TopFluor labeled HDL-NP and Bilayer NP with diluted human serum that the time course of enzymatic activity against the TopFluor HDL-NP is quite rapid, with a significant increase (p=0.00003) in fluorescence within 30 minutes after serum addition to the NPs (FIG. 4), a marked improvement over existing LCAT assays that recommend incubation times of several hours.

FIGS. 5A-5E show (FIG. 5A) synthesis of in situ HDL NP (_{IS}HDL NP) in PBS doped with apoAI, cholesterol and LCAT. (FIG. 5B) Western blot for IS-HDL NP after incubation in PBS doped with increasing amounts of apoAI. (FIG. 5C) Synthesis of IS-HDL NP in human serum. (FIG. 5D) Western blot for apoAI following incubation with increasing amounts of human serum. Positive controls are apoAI alone, 1% human serum, and the conventionally synthesized HDL NP. (FIG. 5E) Confirmation of cholesterol efflux capacity of _{IS}HDL NP following apoAI sequestration.

FIGS. 6A-6C show (FIG. 6A) reaction scheme for measurement of cholesterol esterification on _{IS}HDL NP. Addition of _{IS}HDL NP to a mixture of cholesterol oxidase, horseradish peroxidase, and amplex red reagent generates the highly fluorescent product, resorufin. Addition or omission of cholesterol esterase allows for determination of cholesteryl ester content on _{IS}HDL NP. Measurement of cholesterol on _{IS}HDL NP following incubation with (FIG. 6B) apoAI/cholesterol/LCAT in PBS and (FIG. 6C) human serum.

FIG. 7 is a correlation plot of serum apoAI versus apoAI content on _{IS}HDL NP after serum incubation.

FIG. 8 is a correlation plot of apoAI content in human serum samples versus LCAT activity (i.e. CE) on _{IS}HDL NP.

FIG. 9 is a correlation plot of HDL-mediated cholesterol efflux versus LCAT activity (i.e., CE) on _{IS}NPs after incubation in apoB-depleted human serum.

FIG. 10 shows (A) measurement of HDL function through incubating apoB-depleted human serum with BL-NP to generate _{IS}HDL NP. By isolating the _{IS}HDL NP and running the Amplex Red™ Cholesterol Assay, CE amount can be measured in <1 day. (B) Measurement of cholesterol efflux percentage through the traditional cell-based assay. J774 macrophages are plated on Day 1, followed by incubation with 3H-cholesterol and cyclic AMP on Days 2 and 3, respectively. On Day 4, apoB-depleted serum is added to cells for 4 hours, followed by isolation of the supernatant and quantification of ³H-cholesterol through liquid scintillation counting, thus providing a measurement of cholesterol efflux capacity, and HDL function.

### DETAILED DESCRIPTION

The invention involves, in some aspects, the discovery of a rapid blood test that is capable of measuring one's risk of cardiovascular disease and other health conditions as well as the effects of exercise because of changes in serum protein levels and the activity of an enzyme involved in cholesterol metabolism. The assay is useful, for instance in allowing for tracking of cardiovascular health in response to acute and chronic exercise. The activity of the target protein and enzyme has been reported to reside at a baseline level and then the enzyme activity increases after exercise. The methods of the invention require only a small amount of a biological sample, such as blood (obtained via pinprick) for analysis. The methods are accomplished using synthetic lipid functionalized nanoparticles as a tool to accurately measure protein adsorption and enzyme activity in real time.

The methods can be performed using any standard medical equipment, health monitors (Clinical Applications) or lab equipment that enables the removal of a small blood sample from a patient. It has also been discovered that the assay can be used with wearable devices or other portable devices such as fitness trackers that are modified to enable a small blood draw.

The synthetic nanoparticle useful in the invention allows for greater specificity for tracking the activity of an exercise associated enzyme such as lecithin:cholesterol acyltransferace (LCAT). Existing test kits that measure LCAT levels or activity may have confounding variables in serum testing due to other enzymes such as phospholipase A2 exhibiting activity against the provided substrates. These types of assays are also quite slow (hours). The structure of the lipid nanostructure enables sequestration of a protein (apoAI) that is the activator of LCAT, which improved specificity over existing tests. Further, current fluorescent-based tests do not include an inherent LCAT activator, such as the lipid nanostructure with apoAI bound, whereby specificity of activity can be assessed.

Thus, in aspects, the invention is a blood test for measuring the activity of lecithin:cholesterol acyltransferase (LCAT), utilizing synthetic in situ nanoparticles (NPs) to sequester LCAT activators such as ApoA1 in the blood. The NPs containing sequestered LCAT activators can function as substrates for enzyme activity. A minimal amount of serum is needed for measurement (e.g. single drop of blood or down to one microliter of serum), with test results available within minutes. LCAT is an enzyme whose activity acutely rises after exercise and then the activity is believed to reduce to a lower baseline level. By measuring LCAT activity before and after exercise, for example, one can get an idea as to their “molecular fitness” score.

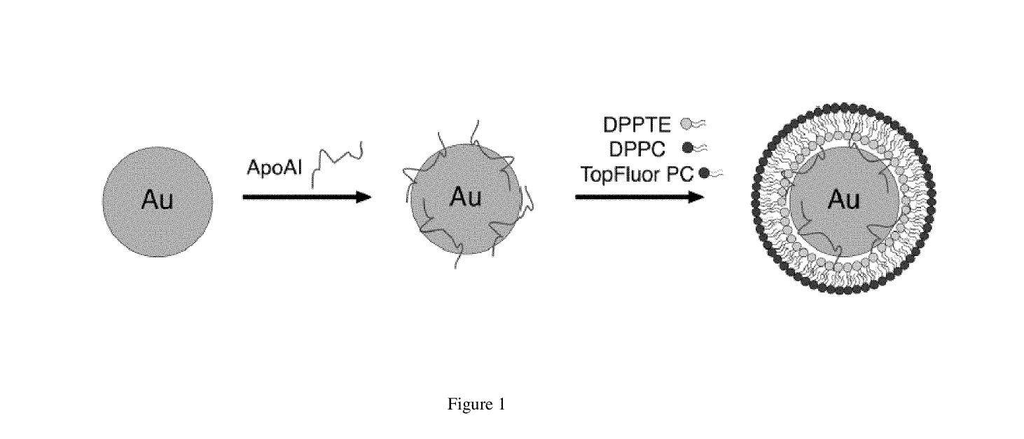

LCAT is known to be activated by apolipoprotein A-1, a protein tightly associated with the surface of the HDL-NP, whereby LCAT esterifies free cholesterol to form cholesteryl ester. The substrates for LCAT are cholesterol and a phospholipid whereby the alkyl tail at the SN2 position of the phospholipid is transferred to the cholesterol —OH group by the enzyme. The NP can be formed using commercially available 5 nm AuNP. In some embodiments ApoA1 may be added to the NP before exposure to the sample and in other embodiments the ApoA1 in the biological sample is simply sequestered by the NP. When the NP is designed with ApoA1 on the surface, apoAI may be added to an aqueous solution of 5 nm AuNP at 5-fold molar excess. In some embodiments, the lipids used are a) 1,2-dipalmitoyl-sn-glycero-3-phosphothioethanol (DPPTE), a thiolated lipid that binds covalently to the surface of the AuNP32 to form the inner leaflet, b) a phosphotidylcholine (PC) lipid with a fluorophore conjugated to the sn2 position of the acyl chain (e.g. TopFluor PC, 1-palmitoyl-2-(dipyrrometheneboron difluoride)undecanoyl-sn-glycero-3-phosphocholine, Avanti Polar Lipids, Inc.) and lastly, c) 1,2-dipalmitoyl-sn-glycero-3-phosphocholine (DPPC) which will form the majority of the outer leaflet of the NP lipid bilayer. FIG. 1 depicts the formation of NP with each component step, with TopFluor PC used as an example fluorescent lipid. The NPs can also be formed in situ by incubating an aqueous solution of 5 nm AuNPs that have a surface monolayer or bilayer of phospholipids with a solution containing apoA-I, such as a biological fluid.

Additional non-limiting examples of fluorescent phospholipids include, without limitation, fatty acid labeled and head group labeled phospholipids. Non-limiting examples of fluorescently labeled phospholipids include cardiolipin labeled with TopFluor®, phosphatidylserine labeled with NBD or Dansyl, and phosphatidylethanolamine labeled with dansyl, pyrene fluorescein, lissamine rhodamine B, NBD, Cy5, Cy5.5, Cy7, etc.

These particles are ideal for at least two reasons. NPs having ApoA1 attached thereto have been previously demonstrated to sequester free cholesterol in a manner similar to native HDL. Also the inclusion of the ApoA1 protein serves as a cofactor to activate LCAT and promote its activity on cholesterol bound to the NP. By incorporating a fluorescent phosphotidylcholine into the outer leaflet of the NP, it has been demonstrated that incubation of fluorescent NP with mixtures of purified LCAT and cholesterol for 24 hours results in an increase in fluorescence. The specificity of the NP for LCAT activity has also been demonstrated. Furthermore, it has been determined that the time course of enzymatic activity against the TopFluor NP is quite rapid, with a significant increase in fluorescence within 30 minutes after serum addition to the NPs, in some assays. Thus, the invention enables a rapid test. In some embodiments the assay may be completed in 5 minutes. In other embodiments the assay may be completed within 10 minutes, 15 minutes, 20 minutes, 25 minutes, 30 minutes, 45 minutes, 60 minutes, 2 hours, 3 hours, 4 hours, 5 hours, 6, hours, or 10 hours.

The invention described herein, in some aspects, is a versatile platform for targeted measurement of LCAT activity using synthetic nanostructures such as nanoparticles (NPs) having a solid core and a lipid shell. The NPs may be used in in vitro assays or may be incorporated into a carrier such as a medical device or wearable device or the NPs can be used as a therapy.

Nanostructures include, for instance, HDL NPs. HDL-NPs are synthesized using a nanoparticle core, such as a gold core, to control size and shape, and modified lipids that harbor an LCAT activator. Synthetic high density lipoprotein nanoparticles have been designed with similar characteristics to natural HDL (the ‘good’ cholesterol).

Alternatively nanostructures may be synthesized using a nanoparticle core, such as a gold core, to control size and shape, and modified lipids that harbor an LCAT activator, but without the addition of LCAT activator. These are referred to simply as NPs. The NPs are designed to interact with the LCAT activator in a biological sample or in a subject in vivo.

Furthermore, cardiovascular disease (CVD) remains the most common cause of death in the developed world and it remains important to assess an individual's risk of developing CVD and then track how specific interventions or lifestyle modifications modify risk. The cholesterol efflux assay (CEA) provides data that predicts the risk for developing cardiovascular diseases or conditions. Although clinically validated, the CEA assay is, among other things, expensive, requires trained personnel, uses radioactive components, and is not amenable for high-throughput or clinical use. Due to the translational challenges of the CEA and to increase the breadth of testing available for HDL function, the assay described herein was developed to capture critical HDL functional parameters and that the data generated correlates with the clinically-validated CEA.

In some aspects, the invention provides a novel method for assessing HDL function. The phospholipid bilayer nanoparticle (BL-NP) sensor platform indirectly measures the amount of apoAI in a sample and LCAT activity through quantification of cholesteryl ester. In some aspects, BL-NPs with an inorganic core are surface-functionalized to spontaneously assemble apoAI from solution to form HDL-like nanoparticles (HDL-NP), which support LCAT activity. BL-NPs rapidly and preferentially adsorb apoAI from pure solutions and human serum according to the abundance of apoAI in the sample. In some embodiments, using serum, NPs formed in situ (_{IS}HDL-NPs) provide apoAI and substrates for LCAT-mediated esterification of cholesterol on the _{IS}HDL-NP. The apoAI on the _{IS}HDL NP is a co-factor of LCAT, while cholesterol and phospholipid on the nanoparticle surface are substrates for LCAT. After isolation of the HDL-NPs, cholesteryl ester can be detected. The levels of cholesteryl ester directly correlate with the apoAI bound by BL-NPs in the sample.

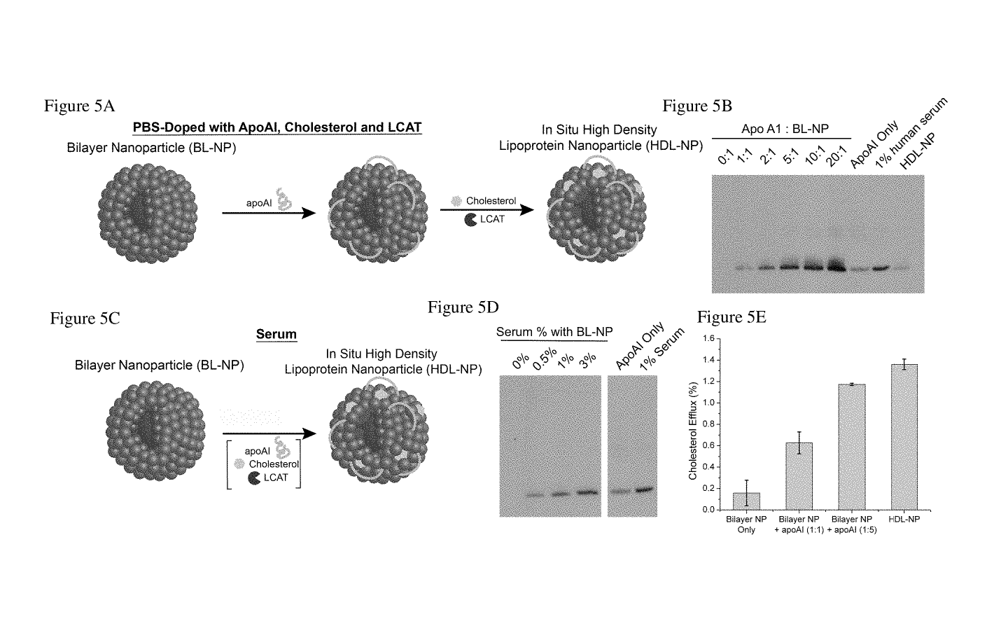

Non-limiting examples of the benefits of the claimed invention include: (1) BL-NPs quantitatively sequester apoAI such that the amount of apoAI that binds to the _{IS}HDL-NPs depends on the apoAI available in the sample (FIGS. 5A-5E); (2) the level of apoAI correlates with the amount of cholesteryl ester bound to _{IS}HDL-NP in serum, thus, demonstrating the potential for the _{IS}HDL NP for detecting, in some aspects, variations in levels of apoAI in serum. And, (3) the cholesteryl ester formed by and sequestered within _{IS}HDL NP significantly correlates to total cholesterol efflux to serum measured using the clinically-validated CEA. Furthermore, the BL-NP biosensor platform provides results in a matter of hours and without the use of radioactive materials contrary to the currently available methods, such as CEA, which provides results over the course of days and uses radioactive materials.

In some aspects, the BL-NP biosensor platform described herein for directly measuring cholesteryl ester detects changes in apoAI and LCAT activity that may result from interventions, such as therapeutic interventions, exercise or dietary modifications.

Thus, the multiple advantages of the BL-NP platform described herein may enable widespread, point-of-care, high-throughput testing of important HDL functional parameters (e.g., apoAI, LCAT activity, etc.) to further patient monitoring.

It has also been discovered according to aspects of the invention that NPs having a minimal structure—composed simply of an inorganic core and a lipid bilayer or monolayer, and not including any apolipoprotein, function as active agents when delivered in vivo. These NPs function similar to high density lipoprotein nanoparticles (HDL NPs), mimic natural spherical HDLs in their shape, size, surface composition (apolipoprotein A1, phospholipids), and have the ability to functionally efflux cholesterol from cells. High-density lipoproteins (HDL) are naturally occurring nanoparticles that assemble dynamically in serum from phospholipids, apolipoproteins, and cholesterol. HDL is involved in reverse-cholesterol transport, and has been epidemiologically correlated with reduced incidences of cardiovascular disease (Asztalos, B. F., Tani, M. & Schaefer, E. J. et al. Metabolic and functional relevance of HDL subspecies. Current Opinion in Lipidology 22, 176-185 (2011); Barter, P. et al. HDL cholesterol, very low levels of LDL cholesterol, and cardiovascular events. N. Engl. J. Med. 357, 1301-1310 (2007). Natural HDL is known to bind Scavenger Receptor type B-1 (SR-B1); SR-B1 mediates uptake of cholesteryl esters and the uptake and efflux free cholesterol.

It was discovered, quite unexpectedly, that the NPs, which did not include ApoA1 could still sequester cholesterol in biological samples. It was surprising because the ApoA1 is an essential component in the process of sequestering cholesterol. Although not being bound by a mechanism, it is believed that the NPs are capable of binding to ApoA1 in serum and other biological samples, effectively forming HDL-NPs in situ or in vivo, which can then sequester cholesterol. The NPs are functioning similar to a pro-drug which is converted into HDL-NPs. Thus, the NPs described herein are therapeutic agents and are useful in the treatment of disorders associated with excess cholesterol.

By administering the NPs described herein, circulating serum HDL levels (e.g., low HDL levels) may be increased. This can provide a promising therapeutic approach to, for example, preventing and potentially reversing atherosclerosis by augmenting reverse cholesterol transport. In other embodiments, compositions and methods described herein may be used to decrease cholesterol or LDL levels (e.g., decrease high cholesterol LDL levels).

Diseases conditions associated with high cholesterol levels which could benefit from the methods described herein include, for example, atherosclerosis, phlebosclerosis or any venous condition in which deposits of plaques containing cholesterol or other material are formed within the intima or inner media of veins, acute coronary syndromes, angina including, stable angina, unstable angina, inflammation, sepsis, vascular inflammation, dermal inflammation, congestive heart failure, coronary heart disease (CHD), ventricular arrythmias, peripheral vascular disease, myocardial infarction, onset of fatal myocardial infarction, non-fatal myocardial infarction, ischemia, cardiovascular ischemia, transient ischemic attacks, ischemia unrelated to cardiovascular disease, ischemia-reperfusion injury, decreased need for revascularization, coagulation disorders, thrombocytopenia, deep vein thrombosis, pancreatitis, non-alcoholic steatohepatitis, diabetic neuropathy, retinopathy, painful diabetic neuropathy, claudication, psoriasis, critical limb ischemia, impotence, dyslipidemia, hyperlipidemia, hyperlipoproteinemia, hypoalphalipoproteinemia, hypertriglyceridemia, any stenotic condition leading to ischemic pathology, obesity, diabetes including both Type I and Type II, ichtyosis, stroke, vulnerable plaques, lower-limb ulceration, severe coronary ischemia, lymphomas, cataracts, endothelial dysfunction, xanthomas, end organ dysfunction, vascular disease, vascular disease that results from smoking and diabetes, carotid and coronary artery disease, regress and shrink established plaques, unstable plaques, vessel intima that is weak, unstable vessel intima, endothelial injury, endothelial damage as a result of surgical procedures, morbidity associated with vascular disease, ulcerations in the arterial lumen, restenosis as a result of balloon angioplasty, protein storage diseases (e.g., Alzheimer's disease, prion disease), diseases of hemostasis (e.g., thrombosis, thrombophilia, disseminated intravascular coagulation, thrombocytopenia, heparin induced thrombocytopenia, thrombotic thrombocytopenic purpura), rheumatic diseases (e.g., multiple sclerosis, systemic lupus erythematosis, sjogren's syndrome, polymyositis/dermatomyositis, scleroderma), neuroligical diseases (e.g., Parkinson's disease, Alzheimer's disease), and subindications thereof.

Additionally the NPs may be used to treat cancer. For example the NPs are able bind to ApoA1 in vivo in order to mimic the interaction between natural HDL and the scavenger receptor type B-1 (SR-B1). Cancer cells which express this receptor—notably lymphomas, prostate cancer, and breast cancer cells are selectively targeted by the NPs. Cytotoxicity has been shown to be higher in lymphoma and epithelial malignancies than towards cardiomyoblasts using HDL-NP.

The shell may have an inner surface and an outer surface, such that the apolipoprotein may be adsorbed on the outer shell and/or incorporated between the inner surface and outer surface of the shell. In some embodiments, the shell comprises one or more cholesterol molecules.

Examples of nanostructures that can be used in the methods are described herein are now described. The structure (e.g., a synthetic structure or synthetic nanostructure) has a core and a shell surrounding the core. In embodiments in which the core is a nanostructure, the core includes a surface to which one or more components can be optionally attached. For instance, in some cases, core is a nanostructure surrounded by shell, which includes an inner surface and an outer surface. The shell may be formed, at least in part, of one or more components, such as a plurality of lipids, which may optionally associate with one another and/or with surface of the core. For example, components may be associated with the core by being covalently attached to the core, physiosorbed, chemisorbed, or attached to the core through ionic interactions, hydrophobic and/or hydrophilic interactions, electrostatic interactions, van der Waals interactions, or combinations thereof. In one particular embodiment, the core includes a gold nanostructure and the shell is attached to the core through a gold-thiol bond.

Optionally, components can be crosslinked to one another. Crosslinking of components of a shell can, for example, allow the control of transport of species into the shell, or between an area exterior to the shell and an area interior of the shell. For example, relatively high amounts of crosslinking may allow certain small, but not large, molecules to pass into or through the shell, whereas relatively low or no crosslinking can allow larger molecules to pass into or through the shell. Additionally, the components forming the shell may be in the form of a monolayer or a multilayer, which can also facilitate or impede the transport or sequestering of molecules. In one exemplary embodiment, shell includes a lipid bilayer that is arranged to sequester cholesterol and/or control cholesterol efflux out of cells, as described herein.

It should be understood that a shell which surrounds a core need not completely surround the core, although such embodiments may be possible. For example, the shell may surround at least 50%, at least 60%, at least 70%, at least 80%, at least 90%, or at least 99% of the surface area of a core. In some cases, the shell substantially surrounds a core. In other cases, the shell completely surrounds a core. The components of the shell may be distributed evenly across a surface of the core in some cases, and unevenly in other cases. For example, the shell may include portions (e.g., holes) that do not include any material in some cases. If desired, the shell may be designed to allow penetration and/or transport of certain molecules and components into or out of the shell, but may prevent penetration and/or transport of other molecules and components into or out of the shell. The ability of certain molecules to penetrate and/or be transported into and/or across a shell may depend on, for example, the packing density of the components forming the shell and the chemical and physical properties of the components forming the shell. The shell may include one layer of material, or multilayers of materials in some embodiments.

The core of the nanostructure whether being a nanostructure core or a hollow core, may have any suitable shape and/or size. For instance, the core may be substantially spherical, non-spherical, oval, rod-shaped, pyramidal, cube-like, disk-shaped, wire-like, or irregularly shaped. The core (e.g., a nanostructure core or a hollow core) may have a largest cross-sectional dimension (or, sometimes, a smallest cross-section dimension) of, for example, less than or equal to about 500 nm, less than or equal to about 250 nm, less than or equal to about 100 nm, less than or equal to about 75 nm, less than or equal to about 50 nm, less than or equal to about 40 nm, less than or equal to about 35 nm, less than or equal to about 30 nm, less than or equal to about 25 nm, less than or equal to about 20 nm, less than or equal to about 15 nm, or less than or equal to about 5 nm. In some cases, the core has an aspect ratio of greater than about 1:1, greater than 3:1, or greater than 5:1. As used herein, “aspect ratio” refers to the ratio of a length to a width, where length and width measured perpendicular to one another, and the length refers to the longest linearly measured dimension.

The core may be formed of an inorganic material. The inorganic material may include, for example, a metal (e.g., Ag, Au, Pt, Fe, Cr, Co, Ni, Cu, Zn, and other transition metals), a semiconductor (e.g., silicon, silicon compounds and alloys, cadmium selenide, cadmium sulfide, indium arsenide, and indium phosphide), or an insulator (e.g., ceramics such as silicon oxide). The inorganic material may be present in the core in any suitable amount, e.g., at least 1 wt %, 5 wt %, 10 wt %, 25 wt %, 50 wt %, 75 wt %, 90 wt %, or 99 wt %. In one embodiment, the core is formed of 100 wt % inorganic material. In some embodiments, the core is 1, nm, 2 nm, 3 nm, 4 nm, 5 nm, 6 nm, 7 nm, 8 nm, 9 nm, 10 nm, 20 nm, 30 nm, 40 nm 50 nm, 60 nm, 70 nm, 80 nm, 90 nm, or 100 nm in diameter. The nanostructure core may, in some cases, be in the form of a quantum dot, a carbon nanotube, a carbon nanowire, or a carbon nanorod. In some cases, the nanostructure core comprises, or is formed of, a material that is not of biological origin. In some embodiments, a nanostructure includes or may be formed of one or more organic materials such as a synthetic polymer and/or a natural polymer. Examples of synthetic polymers include non-degradable polymers such as polymethacrylate and degradable polymers such as polylactic acid, polyglycolic acid and copolymers thereof. Examples of natural polymers include hyaluronic acid, chitosan, and collagen.

Furthermore, a shell of a structure can have any suitable thickness. For example, the thickness of a shell may be at least 10 Angstroms, at least 0.1 nm, at least 1 nm, at least 2 nm, at least 5 nm, at least 7 nm, at least 10 nm, at least 15 nm, at least 20 nm, at least 30 nm, at least 50 nm, at least 100 nm, or at least 200 nm (e.g., from the inner surface to the outer surface of the shell). In some cases, the thickness of a shell is less than 200 nm, less than 100 nm, less than 50 nm, less than 30 nm, less than 20 nm, less than 15 nm, less than 10 nm, less than 7 nm, less than 5 nm, less than 3 nm, less than 2 nm, or less than 1 nm (e.g., from the inner surface to the outer surface of the shell). Such thicknesses may be determined prior to or after sequestration of molecules as described herein.

The shell of a structure described herein may comprise any suitable material, such as a hydrophobic material, a hydrophilic material, and/or an amphiphilic material. Although the shell may include one or more inorganic materials such as those listed above for the nanostructure core, in many embodiments the shell includes an organic material such as a lipid or certain polymers. The components of the shell may be chosen, in some embodiments, to facilitate the binding capacity as well as binding affinity of the therapeutic agent. For example, positively charged head groups in the outer layer can decrease the binding affinity of a therapeutic agent such as doxorubicin, while negatively charged lipid head groups increase the binding affinity of doxorubicin. Changes in the lipid composition of the nanoparticle can not only change the binding affinity between therapeutic agent and the nanostructure, but also the binding capacity of the nanostructure for therapeutic agent. The binding affinity of the nanoparticles may be further altered by including cholesterol (the modulate fluidity of the lipid layer), Poly(styrenesulfonate) (negatively charged polymer for enhanced doxorubicin binding) or DNA (with a doxorubicin binding motif) in the synthesis step.

In some embodiments, 10%, 20%, 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 100% of the organic material has head groups in the outer layer of the shell that are positively charged. In some embodiments, 10%, 20%, 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 100% of the organic material has head groups in the outer layer of the shell that are negatively charged. In some embodiments, 10%, 20%, 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 100% of the organic material has head groups in the outer layer of the shell are neutral.

In one set of embodiments, a structure described herein or a portion thereof, such as a shell of a structure, includes one or more natural or synthetic lipids or lipid analogs (i.e., lipophilic molecules). One or more lipids and/or lipid analogues may form a single layer or a multi-layer (e.g., a bilayer) of a structure. In some instances where multi-layers are formed, the natural or synthetic lipids or lipid analogs interdigitate (e.g., between different layers). Non-limiting examples of natural or synthetic lipids or lipid analogs include fatty acyls, glycerolipids, glycerophospholipids, sphingolipids, saccharolipids and polyketides (derived from condensation of ketoacyl subunits), and sterol lipids and prenol lipids (derived from condensation of isoprene subunits).

In one particular set of embodiments, a structure described herein includes one or more phospholipids. The one or more phospholipids may include, for example, 1,2-Dipalmitoyl-sn-Glycero-3-Phosphothioethanol (DPPTE), phosphatidylcholine (PC), phosphatidylglycerol, lecithin, β, γ-dipalmitoyl-α-lecithin, sphingomyelin, phosphatidylserine, phosphatidic acid, N-(2,3-di(9-(Z)-octadecenyloxy))-prop-1-yl-N,N,N-trimethylammonium chloride, phosphatidylethanolamine, lysolecithin, lysophosphatidylethanolamine, phosphatidylinositol, cephalin, cardiolipin, cerebrosides, dicetylphosphate, dioleoylphosphatidylcholine, dipalmitoylphosphatidylcholine, dipalmitoylphosphatidylglycerol, dioleoylphosphatidylglycerol, palmitoyl-oleoyl-phosphatidylcholine, di-stearoyl-phosphatidylcholine, stearoyl-palmitoyl-phosphatidylcholine, di-palmitoyl-phosphatidylethanolamine, di-stearoyl-phosphatidylethanolamine, di-myrstoyl-phosphatidylserine, di-oleyl-phosphatidylcholine, 1,2-dipalmitoyl-sn-glycero-3-phosphothioethanol, 1,2-dipalmitoyl-sn-glycero-3-phosphocholine (DPPC) and combinations thereof. In some cases, a shell (e.g., a bilayer) of a structure includes 50-200 natural or synthetic lipids or lipid analogs (e.g., phospholipids). For example, the shell may include less than about 500, less than about 400, less than about 300, less than about 200, or less than about 100 natural or synthetic lipids or lipid analogs (e.g., phospholipids), e.g., depending on the size of the structure. In some embodiments, the nanostructure includes 10-100, 20-100, 30-100, 40-100, 50-100, 60-100, 70-100, 80-100 or 90-100 natural or synthetic lipids or lipid analogs (e.g., phospholipids). In certain embodiments, the nanostructure includes 71-95 natural or synthetic lipids or lipid analogs (e.g., phospholipids). In some embodiments, the lipids are on the outer surface of the shell.

Non-phosphorus containing lipids may also be used such as stearylamine, docecylamine, acetyl palmitate, and fatty acid amides. In other embodiments, other lipids such as fats, oils, waxes, cholesterol, sterols, fat-soluble vitamins (e.g., vitamins A, D, E and K), glycerides (e.g., monoglycerides, diglycerides, triglycerides) can be used to form portions of a structure described herein.

A portion of a structure described herein such as a shell or a surface of a nanostructure may optionally include one or more alkyl groups, e.g., an alkane-, alkene-, or alkyne-containing species, that optionally imparts hydrophobicity to the structure. An “alkyl” group refers to a saturated aliphatic group, including a straight-chain alkyl group, branched-chain alkyl group, cycloalkyl (alicyclic) group, alkyl substituted cycloalkyl group, and cycloalkyl substituted alkyl group. The alkyl group may have various carbon numbers, e.g., between C2 and C40, and in some embodiments may be greater than C5, C10, C15, C20, C25, C30, or C35. In some embodiments, a straight chain or branched chain alkyl may have 30 or fewer carbon atoms in its backbone, and, in some cases, 20 or fewer. In some embodiments, a straight chain or branched chain alkyl may have 12 or fewer carbon atoms in its backbone (e.g., C1-C12 for straight chain, C3-C12 for branched chain), 6 or fewer, or 4 or fewer. Likewise, cycloalkyls may have from 3-10 carbon atoms in their ring structure, or 5, 6 or 7 carbons in the ring structure. Examples of alkyl groups include, but are not limited to, methyl, ethyl, propyl, isopropyl, cyclopropyl, butyl, isobutyl, tert-butyl, cyclobutyl, hexyl, cyclochexyl, and the like.

The alkyl group may include any suitable end group, e.g., a thiol group, an amino group (e.g., an unsubstituted or substituted amine), an amide group, an imine group, a carboxyl group, or a sulfate group, which may, for example, allow attachment of a ligand to a nanostructure core directly or via a linker. For example, where inert metals are used to form a nanostructure core, the alkyl species may include a thiol group to form a metal-thiol bond. In some instances, the alkyl species includes at least a second end group. For example, the species may be bound to a hydrophilic moiety such as polyethylene glycol. In other embodiments, the second end group may be a reactive group that can covalently attach to another functional group. In some instances, the second end group can participate in a ligand/receptor interaction (e.g., biotin/streptavidin).

In some embodiments, the shell includes a polymer. For example, an amphiphilic polymer may be used. The polymer may be a diblock copolymer, a triblock copolymer, etc., e.g., where one block is a hydrophobic polymer and another block is a hydrophilic polymer. For example, the polymer may be a copolymer of an α-hydroxy acid (e.g., lactic acid) and polyethylene glycol. In some cases, a shell includes a hydrophobic polymer, such as polymers that may include certain acrylics, amides and imides, carbonates, dienes, esters, ethers, fluorocarbons, olefins, sytrenes, vinyl acetals, vinyl and vinylidene chlorides, vinyl esters, vinyl ethers and ketones, and vinylpyridine and vinylpyrrolidones polymers. In other cases, a shell includes a hydrophilic polymer, such as polymers including certain acrylics, amines, ethers, styrenes, vinyl acids, and vinyl alcohols. The polymer may be charged or uncharged. As noted herein, the particular components of the shell can be chosen so as to impart certain functionality to the structures.

Where a shell includes an amphiphilic material, the material can be arranged in any suitable manner with respect to the nanostructure core and/or with each other. For instance, the amphiphilic material may include a hydrophilic group that points towards the core and a hydrophobic group that extends away from the core, or, the amphiphilic material may include a hydrophobic group that points towards the core and a hydrophilic group that extends away from the core. Bilayers of each configuration can also be formed.

The structures described herein may also include one or more proteins, polypeptides and/or peptides (e.g., synthetic peptides, amphiphilic peptides). In one set of embodiments, the structures include proteins, polypeptides and/or peptides that can increase the rate of cholesterol transfer or the cholesterol-carrying capacity of the structures. The one or more proteins or peptides may be associated with the core (e.g., a surface of the core or embedded in the core), the shell (e.g., an inner and/or outer surface of the shell, and/or embedded in the shell), or both. Associations may include covalent or non-covalent interactions (e.g., hydrophobic and/or hydrophilic interactions, electrostatic interactions, van der Waals interactions).

An example of a suitable protein that may associate with a structure described herein is an apolipoprotein, such as apolipoprotein A (e.g., apo A-I, apo A-II, apo A-IV, and apo A-V), apolipoprotein B (e.g., apo B48 and apo B100), apolipoprotein C (e.g., apo C-I, apo C-II, apo C-III, and apo C-IV), and apolipoproteins D, E, and H. Specifically, apo A1, apo A2, and apo E promote transfer of cholesterol and cholesteryl esters to the liver for metabolism and may be useful to include in structures described herein. Additionally or alternatively, a structure described herein may include one or more peptide analogues of an apolipoprotein, such as one described above. A structure may include any suitable number of, e.g., at least 1, 2, 3, 4, 5, 6, or 10, apolipoproteins or analogues thereof. In certain embodiments, a structure includes 1-6 apolipoproteins, similar to a naturally occurring HDL particle. In some embodiments, the apolipoprotein is a naturally occurring apolipoprotein obtained from a biological sample. In other embodiments, the apolipoprotein is synthetic or recombinant. Of course, other proteins (e.g., non-apolipoproteins) can also be included in structures described herein.

In some embodiments, apolipoprotein B is depleted from a solution, such as serum. Apolipoprotein B can be depleted from solution using methods known to one of ordinary skill in the art. Non-limiting examples of methods for depleting apolipoprotein B include the use of polyethylene glycol, dextran sulfate/magnesium chloride, heparin sodium/manganese chloride, LipoSep immunoprecipitation (Davidson et al., J Lipid Res (2016) 57(4):674-86) and PEG8000. Additional methods for depleting apolipoprotein B known to one of ordinary skill in the art are also contemplated herein.

Optionally, one or more enzymes may also be associated with a structure described herein. For example, lecithin-cholesterol acyltransferase is an enzyme which converts free cholesterol into cholesteryl ester (a more hydrophobic form of cholesterol). In naturally-occurring lipoproteins (e.g., HDL and LDL), cholesteryl ester is sequestered into the core of the lipoprotein, and causes the lipoprotein to change from a disk shape to a spherical shape. Thus, structures described herein may include lecithin-cholesterol acyltransferase to mimic HDL and LDL structures. Other enzymes such as cholesteryl ester transfer protein (CETP) which transfers esterified cholesterol from HDL to LDL species may also be included.

It should be understood that the components described herein, such as the lipids, phospholipids, alkyl groups, polymers, proteins, polypeptides, peptides, enzymes, bioactive agents, nucleic acids, and species for targeting described above (which may be optional), may be associated with a structure in any suitable manner and with any suitable portion of the structure, e.g., the core, the shell, or both. For example, one or more such components may be associated with a surface of a core, an interior of a core, an inner surface of a shell, an outer surface of a shell, and/or embedded in a shell.

A variety of methods can be used to fabricate the nanostructures described herein. Examples of methods are provided in International Patent Publication No. WO/2009/131704, filed Apr. 24, 2009 and entitled, “Nanostructures Suitable for Sequestering Cholesterol and Other Molecules”, which is incorporated herein by reference in its entirety for all purposes.

In some aspects, the invention provides method assays for measuring high density lipoprotein (HDL) function, the method comprising contacting any of the nanostructures described herein that comprises LCAT with a solution or a biological sample, incubating the nanostructure with the solution or biological sample for a time sufficient to sequester one or more apolipoproteins from the solution or biological sample, and detecting the amount of cholesteryl ester formed as measure of the function of HDL. In some aspects, the amount of apolipoprotein available in the solution or biological sample positively correlates with the amount of cholesteryl ester formed and the activity of LCAT.

In some embodiments, the solution is a buffer solution, such as phosphate buffered saline (PBS). In some embodiments, the solution is blood serum obtained from a subject. In some embodiments, the serum is diluted in the solution at a concentration of 0.0001%, 0.001%, 0.01%, 0.05%, 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 40%, 50%, 60%, 70%, 80%, 90% or 100%. In some embodiments, the serum is diluted in buffer, In some embodiments, the serum is diluted in PBS.

In some embodiments, the nanostructure is incubated with a biological sample or solution that includes apolipoproteins to sequester one or more apolipoproteins (e.g., apoAI) from the biological sample or solution for 5 minutes, 10 minutes, 30 minutes, 45 min, 1 hour, 1.5 hours, 2 hours, 3 hours, 4 hours, 5 hours, 10 hours, 15 hours, 20 hours, or 24 hours. In some embodiments, the amount of apolipoprotein sequestered into the nanostructure is detected by methods known to one of ordinary skill in the art to detect protein. Non-limiting examples include Western blotting, enzyme-linked immunosorbent assay, high performance liquid chromatography, liquid chromatography-mass spectrometry, flow cytometry, mass spectrometry, immunoassay, immunoblot etc.

In some embodiments, the assay can be completed in about 4-8 hours. In some embodiments, the assay can be completed in about 6 hours. The amount of time needed to complete the nanoparticle-based assay described herein is significantly shorter (i.e., about six hours) than the amount of time needed to complete the clinically-validated CEA (i.e., about four days). In some embodiments, the assay time can be further reduced using an oxidase-based system or oxidase-based sensing device. The oxidase-based system or oxidase-based sensing device is similar to a glucometer whereupon the biological sample is exposed to the nanostructures whereupon the nanostructures sequester, for instance, apoA-I and cholesterol, which then serves as a substrate for LCAT and subsequent cholesterol esterification. In this case, the sample containing such nanostructures is exposed to a cholesterol oxidase such that the enzyme oxidizes cholesterol and generates hydrogen peroxide. The hydrogen peroxide can be used to generate molecules that can liberate electrons or fluorescence form source moleucules that can be easily measured using hand-held, point-of-use devices.

In some embodiments, the amount of cholesteryl ester formed is detected using an assay, such as a fluorescent assay. For instance, the amount of cholesteryl ester (CE) is determined by measuring the amount of cholesterol in a sample treated with cholesterol esterase (total cholesterol, TC) and the amount of cholesterol in a sample not treated with cholesterol esterase (free cholesterol, FC). Cholesteryl ester is determined by subtracting the free cholesterol from the total cholesterol CE=TC−FC). In the presence of cholesterol esterase, cholesterol esters are hydrolyzed into cholesterol, which is subsequently oxidized by cholesterol oxidase to yield hydrogen peroxide (H₂O₂). H₂O₂ is detected using the enzyme horseradish peroxidase and 10-acetyl-3,7-dihydroxyphenoxazine (Amplex Red™), where the reaction yields the fluorescent product, resorufin. Measured cholesteryl ester amounts directly correlate with the apoAI bound by HDL-NPs in the sample. Other methods that measure cholesteryl ester known to one of ordinary skill in the art are also contemplated herein. The main method to detect cholesteryl esters is described and consists of measuring total cholesterol with and without cholesterol esterase and then subtracting total cholesterol (+ cholesterol esterase) from free cholesterol in the sample (− cholesterol esterase). Non-limiting examples of non-colorimetric assays also contemplated herein include fluorescence-based assays, electrical conductance based assays, densitometry assays, and electrical resistance assays, and spectroscopic assay, like Raman spectroscopy and mass spectrometry.

In some aspects, the invention provides methods for determining the risk for developing a disease or condition in a subject, the method comprising obtaining a sample from the subject, contacting the sample with any of the nanostructures described herein, incubating the nanostructure with the sample in a solution for a time sufficient to sequester one or more apolipoproteins from the sample, detecting the amount of cholesteryl ester formed, comparing the amount of cholesteryl ester formed in the sample with a predetermined value, wherein the predetermined value represents the level of cholesteryl ester formed in a subject free of the disease or condition, and determining whether the subject is at risk of developing the disease or condition by comparing the amount of cholesteryl ester formed in the sample is above, at or below the predetermined value.

In some embodiments, the disease or condition is a cardiovascular disease or condition. The cardiovascular disease or condition can be any cardiovascular disease or condition known to one of ordinary skill in the art. Non-limiting examples of cardiovascular diseases or conditions include heart failure, arteriosclerosis (e.g., atherosclerosis, nonatheromatous arteriosclerosis), valvular disease, inflammation, hypertension (e.g., essential hypertension, renovascular hypertension), cardiomyopathy, myocardial infarction and stroke. For instance, a subject is not considered to be at increased risk of developing a cardiovascular disease or condition if the amount of cholesteryl ester formed in the sample is at or above some predetermined value or that the subject is at risk of developing the cardiovascular disease or condition if the amount of cholesteryl ester formed in the sample is below the predetermined value.

In some embodiments, the disease or condition is a metabolic disease or condition, neurologic disease or condition, an infection, inflammation, a rheumatologic condition, a renal condition, a pulmonary condition, or a reproductive disease or condition known to one of ordinary skill in the art.

In some embodiments, the sample is obtained from a subject by methods known to one of ordinary skill in the art. For example, the sample is a blood sample obtained by a physician, which is processed to isolate the serum component of the blood sample by conventional methods, such as centrifugation.

In some aspects, the invention provides methods for assessing the effect of one or more interventions on a cardiovascular disease or condition in a subject, the method comprising obtaining a sample from the subject, contacting the sample with a nanostructure, incubating the nanostructure with the sample for a time sufficient to sequester one or more apolipoprotein from the sample, detecting the level of cholesteryl ester formed, exposing the subject to one or more treatments and repeating the above-mentioned steps, comparing the levels of cholesteryl ester formed before the one or more interventions with the levels of cholesteryl ester formed after the intervention, and determining whether the one or more interventions improved the cardiovascular disease or condition. For instance, the one or more interventions are considered to improve a cardiovascular disease or condition if the amount of cholesteryl ester formed after the one or more interventions is above the level cholesteryl ester formed before the intervention. On the other hand, the one or more interventions are not considered to improve the cardiovascular disease or condition if the amount of cholesteryl ester formed after the intervention is at or below the level of cholesteryl ester formed before the intervention.

In some embodiments, the intervention is a therapeutic intervention, exercise, or a dietary modification. Non-limiting examples of therapeutic interventions to treat cardiovascular diseases, include but are not limited to nitrates (e.g., nitroglycerine, isosorbide, etc.), beta blockers (e.g., atenolol, metoprolol, nadolol, oxprenolol, pindolol, propranolol, timolol, etc.), alpha blockers (e.g., doxazosin, phentolamine, indoramin, phenoxybenzamine, prazosin, terazosin, tolazoline, etc.), calcium channel blockers (e.g., amlodipine, aranidipine, azelnidipine, barnidipine, benidipine, cilnidipine, clevidipine, isradipine, efonidipine, felodipine, lacidipine, lercanidipine, manidipine, nicardipine, nifedipine, nilvadipine, nimodipine, nisoldipine, nitrendipine, pranidipine, diltiazem, mibefradil, bepridil, fluspirilene, fendiline, etc.), loop diuretics (e.g., bumetanide, ethacrynic acid, furosemide, torsemide, etc.), thiazide diuretics (e.g., epitizide, hydrochlorothiazide, chlorothiazide, bendroflumethiazide, etc.), thiazide-like diuretics (e.g., indapamide, chlorthalidone, metolazone, etc.), potassium-sparing diuretics (e.g., amiloride, triamterene, spironolactone, etc.), beta blockers (e.g., atenolol, metoprolol, nadolol, oxprenolol, pindolol, propranolol, timolol, etc.), alpha blockers (e.g., doxazosin, phentolamine, indoramin, phenoxybenzamine, prazosin, terazosin, tolazoline, etc.), mixed alpha and beta blockers (e.g., bucindolol, carvedilol, labetalol, etc.), dihydropyridines (e.g., amlodipine, felodipine, isradipine, lercanidipine, nicardipine, nifedipine, nimodipine, nitrendipine, etc.), non-dihydropyridines (e.g., diltiazem, verapamil, etc.), ACE inhibitors (e.g., captopril, enalapril, fosinopril, lisinopril, perindopril, quinapril, ramipril, trandolapril, benazepril, etc.), angiotensin II receptor antagonists (e.g., candesartan, eprosartan, irbesartan, losartan, olmesartan, telmisartan, valsartan, etc.), aldosterone receptor antagonists (e.g., eplerenone, spironolactone, etc.), vasodilators (e.g., sodium nitroprusside, hydralazine, etc.), alpha-2 agonists (e.g., clonidine, guanabenz, methyldopa, moxonidine, etc.), adrenergic neuron blockers (e.g., guanethidine, reserpine, etc.), or other therapeutic interventions known to one of ordinary skill in the art. In some embodiments, two or more of the therapeutic intervention, exercise, or dietary modification are administered simultaneously or sequentially.

In some embodiments, the nanoparticles are isolated to measure the levels of cholesteryl ester. In some embodiments, the nanoparticles are isolated by methods known to one of ordinary skill in the art. Non-limiting examples of methods of isolation include centrifugation or the use of magnetism if the nanostructure has a metal core, such as a gold core, or isolation based upon known biological interactions such as antibody-antigen or streptavidin-biotin, or receptor-ligand interactions.

As used herein, a “subject” or a “patient” refers to any mammal (e.g., a human). Examples of subjects or patients include a human, a non-human primate, a cow, a horse, a pig, a sheep, a goat, a dog, a cat or a rodent such as a mouse, a rat, a hamster, or a guinea pig. Generally, the invention is directed toward use with humans.

A “biological sample,” as used herein, is any cell, body tissue, or body fluid sample obtained from a subject. Non-limiting examples of body fluids include, for example, lymph, saliva, blood, serum, urine, and the like. Samples of tissue and/or cells for use in the various methods described herein can be obtained through standard methods including, but not limited to, tissue biopsy, including punch biopsy and cell scraping, needle biopsy; or collection of blood or other bodily fluids by aspiration or other suitable methods.

In some embodiments, the assays described herein may be performed using a wearable device such as a wearable bracelet, anklet, or other device. The device may be designed only to perform the assay of the invention or alternatively may be designed for use with wearable biometric monitoring components (also referred to herein as “biometric tracking devices,” “biometric tracking modules,” “wearable fitness monitors,” or the like). Such devices, which are often designed to be worn as bracelets or wristbands, have a small housing that has a limited area that is in contact with a persons' skin. The devices preferably used in the invention also include a blood extraction element such as a needle or a blade (e.g., a micro-blade) that can extract a drop or two of blood from the wearer of the device and deliver the blood to the NP to initiate the assay.

In other embodiments, a composition is introduced to a subject or a biological sample, and the structures of the composition and/or the subject or biological sample are exposed to assay conditions that can determine a disease or condition of the subject or biological sample. At least a portion of the structures may be retrieved from the subject or biological sample and an assay may be performed with the structures retrieved. The structures may be assayed for the amount and/or type of molecules bound to the structures.

As described herein, the inventive structures may be used in “pharmaceutical compositions” or “pharmaceutically acceptable” compositions, which comprise a therapeutically effective amount of one or more of the structures described herein, formulated together with one or more pharmaceutically acceptable carriers, additives, and/or diluents. The pharmaceutical compositions described herein may be useful for treating cancer or other conditions. It should be understood that any suitable structures described herein can be used in such pharmaceutical compositions, including those described in connection with the figures. In some cases, the structures in a pharmaceutical composition have a nanostructure core comprising an inorganic material and a shell substantially surrounding and attached to the nanostructure core.

The pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream or foam; sublingually; ocularly; transdermally; or nasally, pulmonary and to other mucosal surfaces.

The phrase “pharmaceutically acceptable” is employed herein to refer to those structures, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; autologous human serum or other acceptable human serum for in vivo administration to patients; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

The structures described herein may be orally administered, parenterally administered, subcutaneously administered, and/or intravenously administered. In certain embodiments, a structure or pharmaceutical preparation is administered orally. In other embodiments, the structure or pharmaceutical preparation is administered intravenously. Alternative routes of administration include sublingual, intramuscular, and transdermal administrations.

Pharmaceutical compositions described herein include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, and the particular mode of administration. The amount of active ingredient that can be combined with a carrier material to produce a single dosage form will generally be that amount of the compound which produces a therapeutic effect. Generally, this amount will range from about 1% to about 99% of active ingredient, from about 5% to about 70%, or from about 10% to about 30%.

The inventive compositions suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a structure described herein as an active ingredient. An inventive structure may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol, glycerol monostearate, and non-ionic surfactants; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered structure is moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the structures described herein include pharmaceutically acceptable emulsions, microemulsions, solutions, dispersions, suspensions, syrups and elixirs. In addition to the inventive structures, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions described herein (e.g., for rectal or vaginal administration) may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the body and release the structures.

Dosage forms for the topical or transdermal administration of a structure described herein include powders, sprays, ointments, pastes, foams, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to the inventive structures, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the structures described herein, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a structure described herein to the body. Dissolving or dispersing the structure in the proper medium can make such dosage forms. Absorption enhancers can also be used to increase the flux of the structure across the skin. Either providing a rate controlling membrane or dispersing the structure in a polymer matrix or gel can control the rate of such flux.

Ophthalmic formulations, eye ointments, powders, solutions and the like, are also contemplated as being within the scope of this invention.

Pharmaceutical compositions described herein suitable for parenteral administration comprise one or more inventive structures in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers, which may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the inventive structures may be facilitated by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Delivery systems suitable for use with structures and compositions described herein include time-release, delayed release, sustained release, or controlled release delivery systems, as described herein. Such systems may avoid repeated administrations of the structures in many cases, increasing convenience to the subject and the physician. Many types of release delivery systems are available and known to those of ordinary skill in the art. They include, for example, polymer based systems such as polylactic and/or polyglycolic acid, polyanhydrides, and polycaprolactone; nonpolymer systems that are lipid-based including sterols such as cholesterol, cholesterol esters, and fatty acids or neutral fats such as mono-, di- and triglycerides; hydrogel release systems; silastic systems; peptide based systems; wax coatings; compressed tablets using conventional binders and excipients; or partially fused implants. Specific examples include, but are not limited to, erosional systems in which the composition is contained in a form within a matrix, or diffusional systems in which an active component controls the release rate. The compositions may be as, for example, microspheres, hydrogels, polymeric reservoirs, cholesterol matrices, or polymeric systems. In some embodiments, the system may allow sustained or controlled release of the active compound to occur, for example, through control of the diffusion or erosion/degradation rate of the formulation. In addition, a pump-based hardware delivery system may be used in some embodiments. The structures and compositions described herein can also be combined (e.g., contained) with delivery devices such as syringes, pads, patches, tubes, films, MEMS-based devices, and implantable devices.

Use of a long-term release implant may be particularly suitable in some cases. “Long-term release,” as used herein, means that the implant is constructed and arranged to deliver therapeutic levels of the composition for at least about 30 or about 45 days, for at least about 60 or about 90 days, or even longer in some cases. Long-term release implants are well known to those of ordinary skill in the art, and include some of the release systems described above.

Injectable depot forms can be made by forming microencapsule matrices of the structures described herein in biodegradable polymers such as polylactide-polyglycolide. Depending on the ratio of structure to polymer, and the nature of the particular polymer employed, the rate of release of the structure can be controlled. Examples of other biodegradable polymers include poly(orthoesters) and poly(anhydrides).

When the structures described herein are administered as pharmaceuticals, to humans and animals, they can be given per se or as a pharmaceutical composition containing, for example, about 0.1% to about 99.5%, about 0.5% to about 90%, or the like, of structures in combination with a pharmaceutically acceptable carrier.

The administration may be localized (e.g., to a particular region, physiological system, tissue, organ, or cell type) or systemic, depending on the condition to be treated. For example, the composition may be administered through parental injection, implantation, orally, vaginally, rectally, buccally, pulmonary, topically, nasally, transdermally, surgical administration, or any other method of administration where access to the target by the composition is achieved. Examples of parental modalities that can be used with the invention include intravenous, intradermal, subcutaneous, intracavity, intramuscular, intraperitoneal, epidural, or intrathecal. Examples of implantation modalities include any implantable or injectable drug delivery system. Oral administration may be useful for some treatments because of the convenience to the patient as well as the dosing schedule.

Regardless of the route of administration selected, the structures described herein, which may be used in a suitable hydrated form, and/or the inventive pharmaceutical compositions, are formulated into pharmaceutically-acceptable dosage forms by conventional methods known to those of skill in the art.

The compositions described herein may be given in dosages, e.g., at the maximum amount while avoiding or minimizing any potentially detrimental side effects. The compositions can be administered in effective amounts, alone or in a combinations with other compounds. For example, when treating cancer, a composition may include the structures described herein and a cocktail of other compounds that can be used to treat cancer. When treating conditions associated with abnormal lipid levels, a composition may include the structures described herein and other compounds that can be used to reduce lipid levels (e.g., cholesterol lowering agents).

The phrase “therapeutically effective amount” as used herein means that amount of a material or composition comprising an inventive structure which is effective for producing some desired therapeutic effect in a subject at a reasonable benefit/risk ratio applicable to any medical treatment. Accordingly, a therapeutically effective amount may, for example, prevent, minimize, or reverse disease progression associated with a disease or bodily condition. Disease progression can be monitored by clinical observations, laboratory and imaging investigations apparent to a person skilled in the art. A therapeutically effective amount can be an amount that is effective in a single dose or an amount that is effective as part of a multi-dose therapy, for example an amount that is administered in two or more doses or an amount that is administered chronically.

The effective amount of any one or more structures described herein may be from about 10 ng/kg of body weight to about 1000 mg/kg of body weight, and the frequency of administration may range from once a day to once a month. However, other dosage amounts and frequencies also may be used as the invention is not limited in this respect. A subject may be administered one or more structure described herein in an amount effective to treat one or more diseases or bodily conditions described herein.

An effective amount may depend on the particular condition to be treated. The effective amounts will depend, of course, on factors such as the severity of the condition being treated; individual patient parameters including age, physical condition, size and weight; concurrent treatments; the frequency of treatment; or the mode of administration. These factors are well known to those of ordinary skill in the art and can be addressed with no more than routine experimentation. In some cases, a maximum dose be used, that is, the highest safe dose according to sound medical judgment.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular inventive structure employed, the route of administration, the time of administration, the rate of excretion or metabolism of the particular structure being employed, the duration of the treatment, other drugs, compounds and/or materials used in combination with the particular structure employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the structures described herein employed in the pharmaceutical composition at levels lower than that required to achieve the desired therapeutic effect and then gradually increasing the dosage until the desired effect is achieved.

In some embodiments, a structure or pharmaceutical composition described herein is provided to a subject chronically. Chronic treatments include any form of repeated administration for an extended period of time, such as repeated administrations for one or more months, between a month and a year, one or more years, or longer. In many embodiments, a chronic treatment involves administering a structure or pharmaceutical composition repeatedly over the life of the subject. For example, chronic treatments may involve regular administrations, for example one or more times a day, one or more times a week, or one or more times a month. In general, a suitable dose such as a daily dose of a structure described herein will be that amount of the structure that is the lowest dose effective to produce a therapeutic effect. Such an effective dose will generally depend upon the factors described above. Generally doses of the structures described herein for a patient, when used for the indicated effects, will range from about 0.0001 to about 100 mg per kg of body weight per day. The daily dosage may range from 0.001 to 50 mg of compound per kg of body weight, or from 0.01 to about 10 mg of compound per kg of body weight. However, lower or higher doses can be used. In some embodiments, the dose administered to a subject may be modified as the physiology of the subject changes due to age, disease progression, weight, or other factors.

If desired, the effective daily dose of the active compound may be administered as two, three, four, five, six or more sub-doses administered separately at appropriate intervals throughout the day, optionally, in unit dosage forms. For example, instructions and methods may include dosing regimens wherein specific doses of compositions, especially those including structures described herein having a particular size range, are administered at specific time intervals and specific doses to achieve reduction of cholesterol (or other lipids) and/or treatment of disease while reducing or avoiding adverse effects or unwanted effects.

While it is possible for a structure described herein to be administered alone, it may be administered as a pharmaceutical composition as described above. The present invention also provides any of the above-mentioned compositions useful for diagnosing, preventing, treating, or managing a disease or bodily condition packaged in kits, optionally including instructions for use of the composition. That is, the kit can include a description of use of the composition for participation in any disease or bodily condition, including those associated with abnormal lipid levels. The kits can further include a description of use of the compositions as discussed herein. The kit also can include instructions for use of a combination of two or more compositions described herein. Instructions also may be provided for administering the composition by any suitable technique, such as orally, intravenously, or via another known route of drug delivery.

The kits described herein may also contain one or more containers, which can contain components such as the structures, signaling entities, and/or biomolecules as described. The kits also may contain instructions for mixing, diluting, and/or administrating the compounds. The kits also can include other containers with one or more solvents, surfactants, preservatives, and/or diluents (e.g., normal saline (0.9% NaCl), or 5% dextrose) as well as containers for mixing, diluting or administering the components to the sample or to the patient in need of such treatment.

The compositions of the kit may be provided as any suitable form, for example, as liquid solutions or as dried powders. When the composition provided is a dry powder, the powder may be reconstituted by the addition of a suitable solvent, which may also be provided. In embodiments where liquid forms of the composition are used, the liquid form may be concentrated or ready to use. The solvent will depend on the particular inventive structure and the mode of use or administration. Suitable solvents for compositions are well known and are available in the literature.

The kit, in one set of embodiments, may comprise one or more containers such as vials, tubes, and the like, each of the containers comprising one of the separate elements to be used in the method. For example, one of the containers may comprise a positive control in the assay. Additionally, the kit may include containers for other components, for example, buffers useful in the assay.

As used herein, a “subject” or a “patient” refers to any mammal (e.g., a human), for example, a mammal that may be susceptible to a disease or bodily condition such as a disease or bodily condition associated with abnormal lipid levels. Examples of subjects or patients include a human, a non-human primate, a cow, a horse, a pig, a sheep, a goat, a dog, a cat or a rodent such as a mouse, a rat, a hamster, or a guinea pig. Generally, the invention is directed toward use with humans. A subject may be a subject diagnosed with a certain disease or bodily condition or otherwise known to have a disease or bodily condition. In some embodiments, a subject may be diagnosed as, or known to be, at risk of developing a disease or bodily condition.

### EXAMPLES

### Example 1

An assay using previously developed synthetic high-density nanoparticles (HDL-NP) as substrates for the enzyme lecithin:cholesterol acyltransferase (LCAT) was performed. HDL-NP was formed using commercially available 5 nm AuNP. apoAI was added to an aqueous solution of 5 nm AuNP at 5-fold molar excess and allowed to bind to the nanoparticle surface for 1 hour with gentle agitation at room temperature. Following this incubation period, a mixture of ethanol (20%, v/v) and lipids were added to the solution at a 250-fold molar excess with respect to the concentration of gold nanoparticles to form the lipid bilayer around the AuNP core. The lipids used were a) 1,2-dipalmitoyl-sn-glycero-3-phosphothioethanol (DPPTE), a thiolated lipid that binds covalently to the surface of the AuNP32 to form the inner leaflet, b) a phosphotidylcholine (PC) lipid with a fluorophore conjugated to the sn2 position of the acyl chain (e.g. TopFluor PC, 1-palmitoyl-2-(dipyrrometheneboron difluoride)undecanoyl-sn-glycero-3-phosphocholine, Avanti Polar Lipids, Inc.) and lastly, c) 1,2-dipalmitoyl-sn-glycero-3-phosphocholine (DPPC) which forms the majority of the outer leaflet of the HDL-NP lipid bilayer. Formation of the lipid bilayer was allowed to occur overnight at room temperature with gentle shaking. Following overnight incubation, the HDL-NP was purified by centrifugation at 15,000×g for 50 minutes, repeated three times to ensure removal of unreacted surface components and ethanol. For larger batches of nanoparticles (>10 ml of initial 5 nm AuNP solution), the centrifugation steps may be omitted and nanoparticles may be processed using a tangential flow filtration system to filter and concentration the final solution of HDL-NP.

FIG. 1 depicts the formation of HDL-NP with each component step, with TopFluor PC used as an example fluorescent lipid.

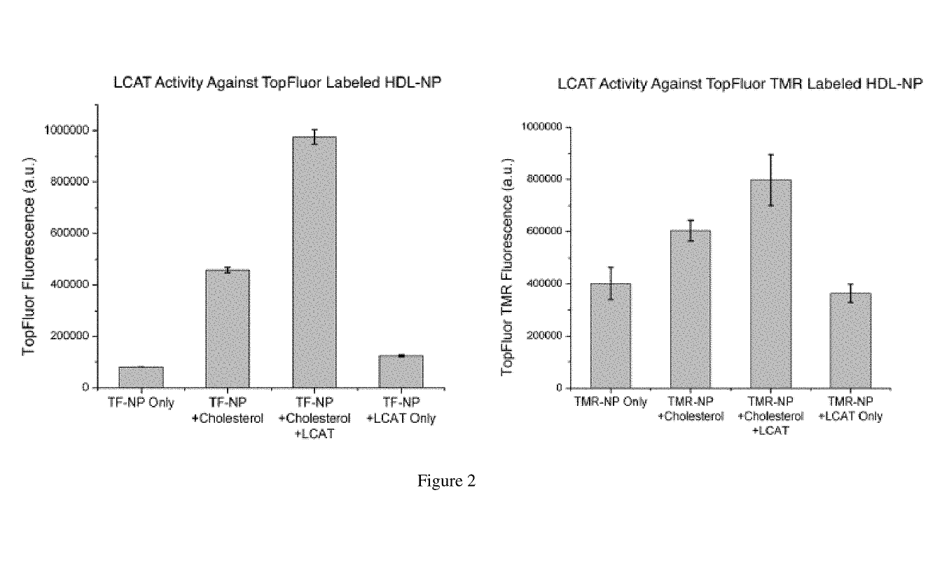

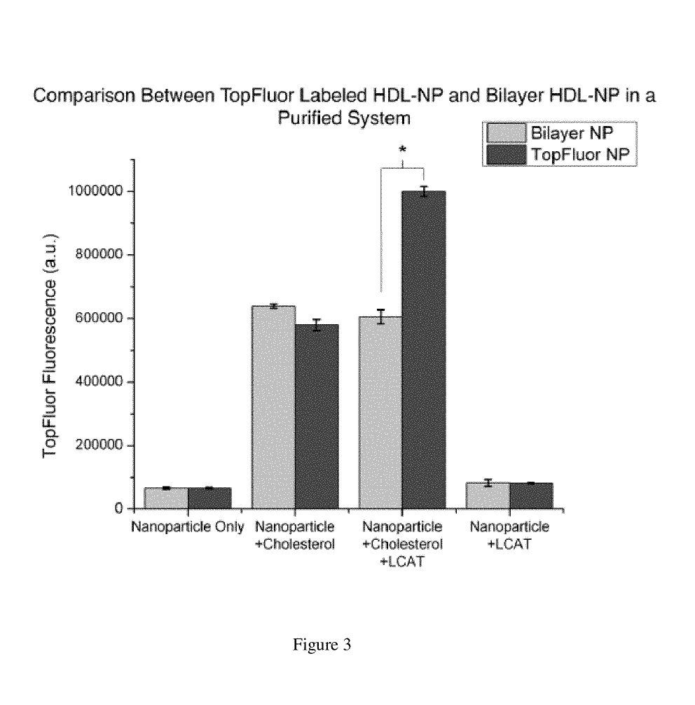

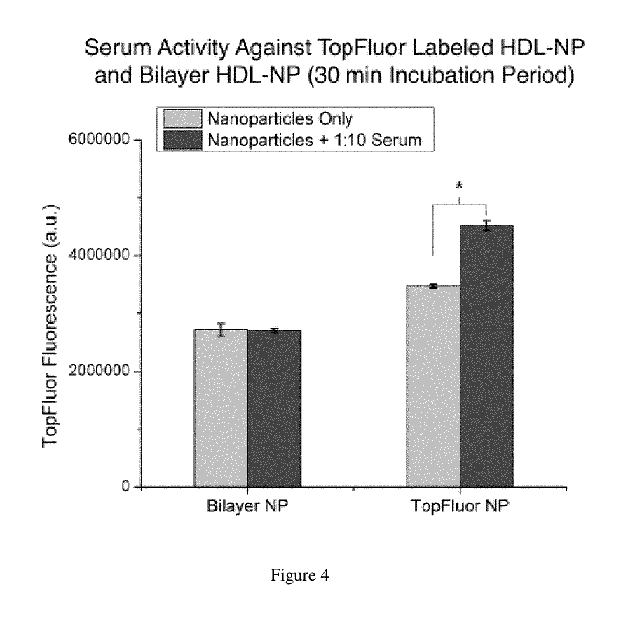

By incorporating a fluorescent phosphotidylcholine into the outer leaflet of the HDL-NP, it has been demonstrated that incubation of fluorescent HDL-NP with mixtures of purified LCAT and cholesterol for 24 hours results in an increase in fluorescence using both TopFluor labeled PC (FIG. 2, left) and TopFluor TMR labeled PC (FIG. 2, right). Utilizing a variant of the TopFluor-loaded HDL-NP without ApoAI, termed the Bilayer NP, the specificity of the HDL-NP for LCAT activity has been demonstrated. FIG. 3 demonstrates that in the presence of cholesterol and LCAT, the fluorescent signal from TopFluor HDL NP is significantly increased over the Bilayer NP group (p=0.00012), and that Bilayer NPs loaded with cholesterol show no significant change in fluorescent signal with or without LCAT (p=0.22). Furthermore, it has been determined through incubations of TopFluor labeled HDL-NP and Bilayer NP with diluted human serum that the time course of enzymatic activity against the TopFluor HDL-NP is quite rapid, with a significant increase (p=0.00003) in fluorescence within 30 minutes after serum addition to the NPs (FIG. 4), a marked improvement over existing LCAT assays that recommend incubation times of several hours. No significant difference in fluorescence was observed between TopFluor Bilayer NPs incubated with serum vs. Top Fluor Bilayer NPs alone for the 30-minute duration of this experiment, once again illustrating the importance of ApoAI in imparting LCAT specificity to the sensor. The increased fluorescent signal may result from an increase in the distance between the fluorescent molecule (TopFluor) and the nanoparticle that provides quenching of the fluorescence when in close proximity, but as cholesterol binds and the cholesterol is esterified with the fluorescent alkyl tail lipid at the SN2 position of the TopFluor, the resulting fluorescently labeled cholesteryl ester further drives the TopFluor Phospholipid away from the core gold nanoparticle as well as the cholesteryl ester moiety being positioned such that the fluorophore is positioned further from the gold nanoparticle core.

### Example 2: A Nanoparticle-Based Assay for Measuring HDL Function Through Apolipoprotein AI Adsorption and Lecithin: Cholesterol Acyl Transferase Activity with Correlation to the Cholesterol Efflux Assay

Measures of LDL- and HDL-cholesterol are used to estimate cardiovascular disease (CVD) risk. Lowering “bad” LDL-C has proved useful in preventing CVD; however, less success has been realized in increasing “good” HDL-C. Recently, data demonstrate that measuring HDL function, versus HDL-C, may provide data that is more useful. For instance, the results of the cholesterol efflux assay (CEA), which measures the ability of apoB-depleted human serum to remove cholesterol from cultured macrophages, are significantly correlated with reduced prevalence of CVD and independently predict CVD. Unfortunately, the complexity, laborious nature, and time and expense of the CEA limit its use. In an effort to make HDL function testing more accessible, 5 nm diameter gold nanoparticles (AuNPs) surface-functionalized with a bilayer of phospholipids (BL-NPs) were utilized to spontaneously assemble apoAI from solution to form HDL-like nanoparticles (HDL NP), which support LCAT activity. BL-NPs rapidly and preferentially adsorb apoAI from pure solutions and human serum according to the abundance of apoAI in the sample. Using serum, in situ formed HDL NPs (_{IS}HDL NPs) provide apoAI and substrates for LCAT-mediated esterification of cholesterol on the HDL NP. After isolation of the nanoparticles, a simple colorimetric assay for free cholesterol (FC) and total cholesterol (TC) [i.e. FC+cholesteryl ester (CE)] was performed. Measured CE amounts directly correlate with the apoAI bound by BL-NPs in the sample. Furthermore, because apoAI and LCAT are critical factors mediating cholesterol efflux measured in the CEA, the correlation between CE obtained with the BL-NP assay and efflux from the CEA was measured using apoB-depleted serum. Data reveal significant correlation. Thus, the multiple advantages of the NP assay may enable widespread, point-of-care, high-throughput testing of HDL parameters, like apoAI and LCAT activity, for comparison with the CEA and further patient monitoring.

### Introduction

High-density lipoproteins (HDL) are dynamic cholesterol carrying nanoparticles in serum. Historically, measuring HDL-cholesterol (HDL-C) provided clinicians with a measure of cardiovascular disease (CVD) fitness such that increased HDL-C suggested reduced prevalence and risk.(1) However, increasing HDL-C in serum has not conclusively proven therapeutically useful(2), and attention has turned toward measuring and perhaps enhancing cardioprotective HDL functions. Among other functions, HDLs are believed to reduce cardiovascular disease (CVD) risk through process known as reverse cholesterol transport (RCT) where free cholesterol (FC) is removed from peripheral tissues and, ultimately, delivered to the liver for excretion. Three critical steps of RCT by HDLs include: 1) HDL engagement of lipid-laden macrophages and efflux of FC from them, 2) FC in HDL is esterified to cholesteryl ester (CE) by lecithin:cholesterol acyl transferase (LCAT) that enables CEs to pack into the core of progressively more cholesterol-rich HDL particles, and 3) Cholesterol-rich HDLs deliver cholesterol to the liver for excretion in feces. The ability of HDLs to uptake, esterify, and deliver cholesterol is dictated by the presence of the HDL-defining apolipoprotein A-I (apoAI).(3) Because of the clinical need, a diagnostic test that measures HDL function may be critically enabling.

An ex vivo approximation of RCT, called the cholesterol efflux assay (CEA), involves the use of J774 cells (murine macrophage cell line) inundated with radiolabeled ³H-cholesterol, and treated with cyclic AMP (cAMP) to increase expression of the ABCA1 receptor.(4-9) Data show that the cholesterol passed from ABCA1 to HDLs significantly contributes to atheroprotective efflux from macrophages.(10) Through the addition of apoB-depleted serum, efflux of cholesterol (e.g. ³H-FC+³H-CE) from the macrophages to HDLs in the matrix can be quantified.(6) The results from the CEA were recently shown to be a superior marker of reduced CVD risk and predicts CVD even when controlling for all other known risk factors.(6, 7) Clearly, issues with the CEA include that the assay requires: several days to complete, cultured macrophages, the use of radiolabeled or fluorescent cholesterol, experienced personnel, is expensive, not amenable to high-throughput required for broad clinical use, and there is significant variability in the assay results across laboratories.(11) Identifying new technologies to measure HDL function would clearly be of tremendous benefit to obtain a better understanding of the correlations between HDL functions and cardiovascular disease, especially in the context of lifestyle modifications and/or medical therapies that modulate HDL function(s).(12)

Several groups (13-15) have previously described the synthesis and applications of bio-inspired high-density lipoprotein-like nanoparticles (HDL NP). The present group uses a 5 nm gold nanoparticle core that serves as a scaffold for the assembly of 2-4 copies of apolipoprotein AI (apoAI) and a phospholipid bilayer.(16-18) The HDL NP constructs are ˜13 nm in diameter and consist of 3±1 apoAI molecules and 83±12 phospholipids in the outer leaflet of the HDL NP membrane, which compares favorably to the reported values for native, mature spherical HDL. Prior work demonstrated that HDL NPs actively efflux cholesterol through all known receptors for HDLs (19) and support LCAT activity.(20) Interestingly, it was demonstrated that the spontaneous assembly of apoAI on HDL NPs required that the gold nanoparticle template be ˜5-6 nm in diameter.(21) Even a slight increase in the nanoparticle size resulted in reduced apoAI assembly and drastically reduced function with regard to binding cholesterol and cellular cholesterol efflux. Thus, 5 nm diameter AuNPs surface functionalized with PLs provides a platform for specific sequestration of apoAI and LCAT activity. For biosensor assays, because of the core gold nanoparticle, a plethora of rapid isolation and readout options are available for assay development.(22-30)

Ultimately, the amount of TC (e.g. ³H-FC+³H-CE) effluxed from cultured macrophages to HDL acceptors and measured by the CEA critically depends upon the amount of apoAI in the apoB-depleted serum sample and cholesterol esterification by LCAT in the serum to maintain a gradient of FC flux from the cells to the HDL acceptors in the sample.(10, 31) New technologies to approximate the results of the CEA should be responsive to these parameters. Because apoAI controls the biosynthesis of native HDLs ranging in size range from 7-14 nm, and the data show that apoAI tightly binds to 5 nm diameter AuNPs surface-functionalized with phospholipids to form HDL NP(21), it was hypothesized that simply adding the 5 nm diameter AuNP surface-functionalized with PLs to serum would enable stable binding of apoAI and the in situ formation of HDL nanoparticles (_{IS}HDL NP). Upon apoAI binding, the HDL NP serves as a template for passively binding FC and supports LCAT activity.(18) The use of the _{IS}HDL NP for assaying LCAT activity is ideal, as HDL NP provides apoAI (the primary activator of LCAT in serum), cholesterol, and a donor pool of PLs from the outer leaflet of the nanoparticle membrane for transesterification of acyl chains to cholesterol. Taken together, the amount of CE formed on _{IS}HDL NPs is a surrogate for parameters that dictate output from the CEA (i.e. apoAI and LCAT), and it was hypothesized that results obtained from the two assays would significantly correlate. Because the BL-NP assay does not require cells or radiolabeled cholesterol, can be done at minimal cost, is rapid (hours vs days), and is amenable to high-throughput, even point-of-care, automation it may well provide a next generation assay to track HDL function as a promising surrogate for the CEA assay.

### Materials and Methods

Synthesis of Phospholipid Bilayer Nanoparticles (BL-NP):

The BL-NP were synthesized by surface-functionalizing 5 nm diameter gold nanoparticles (AuNP, Ted Pella, Redding, Calif.) with phospholipids (250-fold molar excess relative to the AuNP concentration) in a 20% ethanol/80% water solution, as described previously.(21) The PLs used were: a) 1,2-dipalmitoyl-sn-glycero-3-phosphoethanolamine-N-[3-(2-pyridyldithio)propionate] (PDP-PE, Avanti Polar Lipids, Alabaster, Ala.), a disulfide-containing lipid that binds covalently to the surface of the AuNP (32) to form the inner leaflet, and b) 1,2-dipalmitoyl-sn-glycero-3-phosphocholine (DPPC, Avanti Polar Lipids, Alabaster, Ala.) which forms the majority of the outer leaflet of the BL-NP PL bilayer(16, 32). Formation of the PL bilayer was allowed to occur overnight at room temperature with gentle shaking. Following overnight incubation, the BL-NPs were purified using a Kros-Flo tangential flow filtration system (Spectrum Laboratories) to remove any excess PLs, ethanol, and to concentrate the BL-NP.

Binding of Apo AI to BL-NP and Assessment of LCAT Activity:

For experiments in a “pure system”, apoAI in PBS (MyBioSource, San Diego, Calif.) was added to BL-NP (250 nM, final) at ratios of 0:1, 1:1, 2:1, 5:1, 10:1, and 20:1 (M apoAI:M BL-NP) and allowed to incubate with BL-NP for 1 hour at 37° C. to form _{IS}HDL NP. The _{IS}HDL NP were purified using centrifugation at 15,000×g for 50 minutes, repeated three times to ensure removal of unbound apoAI. For LCAT activity determination, 10 μg cholesterol (Sigma-Aldrich, St. Louis, Mo.) and 2.5 μL of 10 nM LCAT (MyBioSource, San Diego, Calif.) were added to the solutions and allowed to incubate for 1 hour at 37° C. Once again, the nanoparticles were similarly separated from excess cholesterol and LCAT using centrifugation. For experiments in serum, serum at 0%, 0.5%, 1%, 3% was added to 250 nM BL-NP (final, 100 μL final volume) and allowed to incubate at 37° C. for 1 hour to allow for apoAI and cholesterol binding to the nanoparticles and for LCAT to esterify cholesterol. Once again, nanoparticles were purified from serum by centrifugation, as above.

Determination of apoAI binding to the BL-NP was accomplished by Western Blot and ELISA. Following purification of the, 20 μL of 100 nM _{IS}HDL NPs was incubated with 4 μl 0.05 M I₂ and 6 μl of 4× loading buffer (BioRad) for 1 hour at 4° C. The samples were then boiled for 8 minutes and spun at 15,900×g for 30 minutes. 25 μl of the supernatant was loaded into a 4-20% Tris-HCl precast gel (BioRad), and run for 32 minutes at 200V. The gel was transferred to PVDF membrane (60V for 90 minutes; BioRad) which was subsequently blocked for 30 minutes at room temperature in 5% blocker in Tris-buffered saline containing 0.1% Triton X-100 (TBST). The apoAI antibody (Abeam) was added to the membrane at a 1:1000 dilution and incubated overnight at 4° C. After rinsing in TBST, the secondary antibody (BioRad) was added at a 1:2000 dilution. Finally, the blot was developed using the ECL developer kit (GE). For controls, apoAI was loaded at 10 μg/ml, serum was loaded at a 1% dilution, and finally 25 nM pre-synthesized HDL-NP were processed as described above for _{IS}HDL NPs and loaded into the gel.

For quantification of apoAI binding on the _{IS}HDL NP using ELISA, a human apoAI ELISA kit (Abeam, Boston, Mass.) was utilized per the instructions provided by the manufacturer.

Measurement of CE on _{IS}HDL NPs:

An Amplex Red Cholesterol Assay (Thermo Fisher Scientific, Waltham, Mass.) was utilized to determine the amount of total cholesterol (TC) in the sample as per the instructions provided by the manufacturer. The only amendment to the protocol was the omission of cholesterol esterase for a subset of samples to enable quantification of the total amount of cholesterol esters in the sample for determination of LCAT activity. Briefly, in the presence of cholesterol esterase, the assay works by hydrolyzing cholesterol esters into cholesterol which is subsequently oxidized by cholesterol oxidase to yield hydrogen peroxide (H₂O₂). The H₂O₂ is detected using horseradish peroxidase and 10-acetyl-3,7-dihydroxyphenoxazine (Amplex Red), where the combination results in the production of the fluorescent product, resorufin. Resorufin was detected in a plate reader at an excitation/emission of 560 nm and 590 nm, respectively. The amounts of cholesterol in each sample were obtained by comparison to a standard cholesterol calibration curve. For determination of cholesterol ester (CE), the total quantified cholesterol in samples with (total cholesterol, TC) and without (free cholesterol, FC) cholesterol esterase were used, and CE=TC−FC.

Cholesterol Efflux Assay:

The cholesterol efflux assay was carried out as described previously. (19, 21) Briefly, J774 mouse macrophages were plated at a density of 1.5×10⁵ cells/well in a 24 well plate, and allowed to adhere overnight. The next day the cells were labeled with 2 μCi/mL of ³H-cholesterol in RPMI, 5% FBS, and were treated with ACAT inhibitor (Sandoz 58-035, 2 μg/mL; Sigma Aldrich). Following 24 hr labeling, the HDL receptor ABCA1 was upregulated by overnight incubation with 8-(4-Chlorophenylthio)adenosine 3′,5′-cyclic monophosphate (cAMP, 0.3 mM; Sigma Aldrich) in RPMI with 0.2% BSA and ACAT inhibitor. To obtain apoB-depleted human serum, single donor human serum samples (innovative Research, Novi, Mi, USA) were mixed with a solution of 20% polyethylene glycol (PEG, average MW=8000), pH=8, at a ratio of 2.5:1 (v/v) serum:PEG. The serum samples were incubated for 20 minutes at room temperature, and then centrifuged at 12,700×g for 30 minutes at 4° C. The supernatant was then diluted in MEM containing 1% HEPES, 2 μg/mL ACAT inhibitor, and 0.03 mM cAMP, and added to the J774 cells. Following 4 hr incubation, the culture media was removed, filtered, and run on a liquid scintillation counter (countsₛᵤₚₑᵣₙₐₜₐₙₜ). The total cellular ³H-cholesterol was quantified by extracting the ³H-cholesterol from J774 cells at the start of the incubation (TO) using isopropanol, followed by liquid scintillation counting (countsₜₒₜₐₗ). The background, free diffusion of cholesterol out of the cells without any acceptor present (counts_{background}) was subtracted from all countsₛᵤₚₑᵣₙₐₜₐₙₜ and the results were divided by countsₜₒₜₐₗ and multiplied by 100 to yield the percent cholesterol efflux: % HDL Efflux=[(countsₛᵤₚₑᵣₙₐₜₐₙₜ−counts_{background})/countsₜₒₜₐₗ)]×100.

Statistics:

All statistical tests were performed on R, version 3.0.1. For determination f correlation between measurements, the Pearson's product-moment correlation test was utilized. P<0.05 denotes statistical significance, and error bars denote standard error of mean.

### Results

Sequestration of ApoAI on Phospholipid Bilayer Nanoparticles:

Following 1 hour of incubation in either an apoAI mixture in PBS or dilute serum, BL-NPs were isolated and purified using centrifugation and probed for apoAI content through Western blotting (FIGS. 5A-5E). These results demonstrate a dose-dependent increase in apoAI, confirming the ability of BL-NP to sequester apoAI from PBS and highly dilute serum, essentially accomplishing an in situ synthesis of HDL NP (_{IS}HDL NP). Also, the amount of apoAI bound by the BL-NP is in dose-response to the amount of apoAI in the sample (FIGS. 5A-5E). The activity of _{IS}HDL NPs with different amounts of apoAI to efflux cholesterol was confirmed using the cholesterol efflux assay (FIG. 5E) with comparison to traditionally synthesized HDL NPs (1:5 ratio of AuNP:apoAI). Data show that _{IS}HDL NP and HDL NP similarly effluxed cholesterol from cultured cells [1.17% efflux for _{IS}HDL NP (1:5) vs. 1.36% efflux for HDL NP].

Detection of LCAT Activity as a Function of apoAI Adsorption:

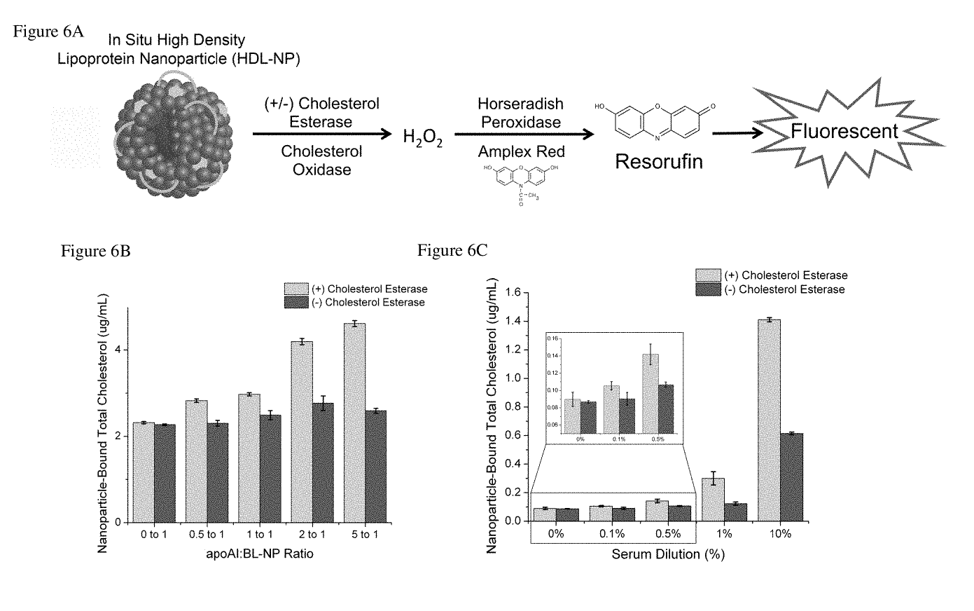

Next, it was determined whether the sequestration of apoAI at increasing concentrations in the incubation medium would result in a concomitant increase in detected TC and CE on BL-NP after exposure to LCAT (FIGS. 6A-6C). It was determined that increasing amounts of apoAI sequestered from PBS resulted in increased CE bound to the _{IS}HDL NP (FIG. 6B). Next, incubating BL-NP with increasing amounts of dilute serum revealed a progressive increase in TC and CE as the amount of serum was increased. Ultimately, serum concentrations of 1% (i.e. 1 μl of serum in 100 μl of 250 nM BL-NP) was deemed adequate for well-defined activity of LCAT on _{IS}HDL NP (FIG. 6C).

Adsorption of ApoAI and CE to BL-NP as a Function of Serum apoAI:

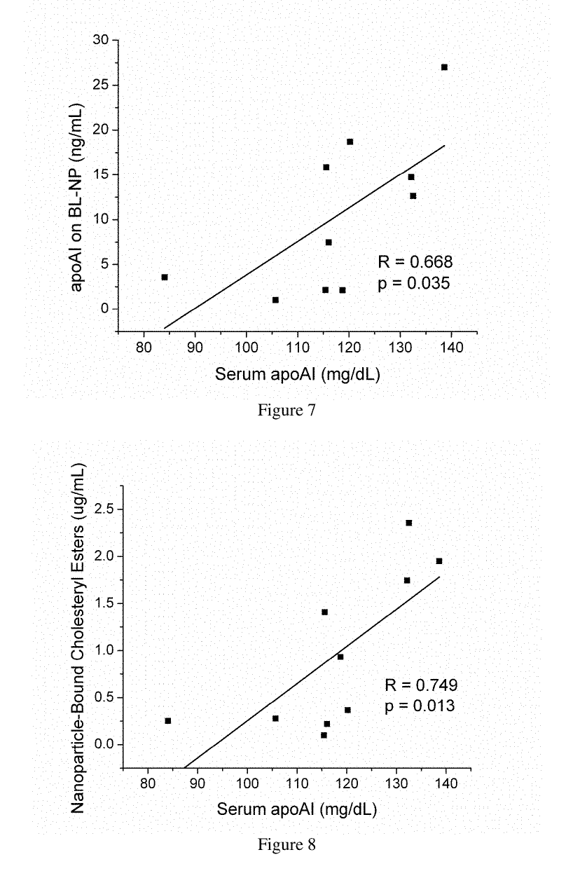

Next, it was sought to assess whether BL-NP could sequester apoAI from serum samples with natural apoAI variation and whether increasing apoAI would correlate with increased CE, hence LCAT activity. Commercially available serum samples from 10 de-identified human donors were purchased (Table 1) and each serum sample was characterized for apoAI, total cholesterol, and HDL-C. Using an ELISA for apoAI, the ability of BL-NP to sequester variable amounts of apoAI from the serum samples was quantified. The results in FIG. 7 depict the correlation plot between the total amount of apoAI in each of the serum samples and the amount of apoAI detected on _{IS}HDL NP following a 1 hour incubation of BL-NP with 1% serum. These data demonstrate a significant positive correlation (R=0.668, p=0.035).

TABLE 1
Characterization data for ten commercially obtained serum samples.

| | 1 | 2 | 3 | 4 | 5 | 6 | 7 | 8 | 9 | 10 |
|---|---|---|---|---|---|---|---|---|---|---|
| Gender | F | M | M | F | M | F | M | M | M | F |
| Ethnicity | C | AA | H | C | C | AA | C | AA | C | H |
| Age (years) | 51 | 24 | 27 | 45 | 28 | 29 | 24 | 29 | 38 | 18 |
| [apoA1] | 116 | 84 | 106 | 120 | 115 | 139 | 132 | 116 | 133 | 119 |
| (mg/dL) | | | | | | | | | | |
| Total | 171 | 143 | 186 | 169 | 155 | 163 | 171 | 153 | 176 | 166 |
| Cholesterol | | | | | | | | | | |
| (mg/dL) | | | | | | | | | | |
| HDL-C | 20 | 25 | 71 | 39 | 49 | 43 | 56 | 52 | 98 | 32 |
| (mg/dL) | | | | | | | | | | |
| CE | 0.21 | 0.25 | 0.27 | 0.36 | 0.09 | 1.95 | 1.74 | 1.4 | 2.35 | 0.93 |
| (ug/mL) | | | | | | | | | | |

Furthermore, the amount of CE associated with _{IS}HDL NP following a 1 hour incubation period with 1% serum was measured. The results indicate that apoAI levels in serum are correlated with the number of CEs detected on the _{IS}HDL NP (R=0.729, p=0.016, FIG. 8).

BL-NP as a Platform for Measurements of Cholesterol Efflux Capacity:

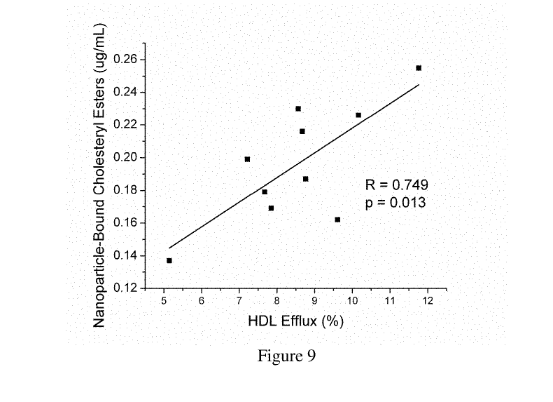

Finally, the relationship between cholesterol esterification on the BL-NP to the conventional CEA method of measuring HDL cholesterol efflux was sought to be investigated. In the CEA, apoB-depleted serum is traditionally used and is generated through the addition of PEG8000 to deplete the serum of apoB-containing lipoproteins. ApoB-depleted serum samples from the 10 individuals in the BL-NP based assay were utilized and—a positive correlation between the CE content of the _{IS}HDL NP and cholesterol efflux obtained using the CEA was found (R=0.749, p=0.013, FIG. 9).

### Discussion

As CVD remains the most common cause of death in the developed world, it continues to be important to assess an individual's risk of developing CVD and then track how specific interventions or lifestyle modifications modify risk. Historically, measures of HDL-C, termed “good cholesterol”, were viewed as reducing CVD risk.(1, 33) However, more recently spot measurements of HDL-C have been called into question due to the failures of therapeutics aimed at increasing HDL-C.(34) As such, measures of HDL function, most specifically using the CEA, have been shown to be powerfully effective at measuring CVD risk.(6, 7, 9, 35) Furthermore, and more broadly, other technologies aimed at measuring other HDL functions beyond cholesterol efflux, are garnering interest to better explain and measure the multitude of HDL-specific factors that may reduce risk.(12) Due to the translational challenges of the CEA and to increase the breadth of testing available for HDL function, it was sought to develop an assay that captures critical HDL functional parameters and the data would correlate with the clinically validated CEA.

The BL-NP sensor platform provides the opportunity to indirectly measure the amount of apoAI in a sample and LCAT activity through quantification of CE. Furthermore, the CE bound to _{IS}HDL NP directly correlates with data obtained using the CEA. Before highlighting the main advantages, it is worthwhile to discuss conceptual limitations of the assay. First, the FC and CE measured in the BL-NP assay does not come from receptor-mediated removal of cholesterol from macrophages. Thus, the possibility exists that the BL-NP represents an enabling substrate for apoAI that would, otherwise, not efflux cholesterol from macrophages whereupon the BL-NP assay would generate a false positive result. The opposite may be true, where a variant of apoA1 may not bind to the BL-NP, but is active in engaging macrophages to support cholesterol efflux. Despite the low likelihood, certainly screening large numbers of biochemically characterized serum samples with direct comparisons to the CEA is required to appreciate the full gamut. Further, certain apoA1 proteins may bind to the BL-NP, but not support LCAT activity. In fact, apoAI variants that do not support LCAT activity are known.(36, 37) Interestingly, the BL-NP assay would identify these individuals as their TC and FC values would consistently be the same with no measured CE. As in the cases mentioned above, further studies are required.

Finally, the data using the BL-NP technology to measure HDL functions are significant for three main reasons: 1) BL-NPs provide a substrate that allows for the quantitative sequestration of apoAI such that the extent of apoAI bound to the _{IS}HDL NPs is dependent on the total amount of apoAI in the medium (FIGS. 5A-5E). The _{IS}HDL NP serves as a specific substrate for LCAT activity due to the presence of the apoAI co-factor and substrates, C and PL, on the nanoparticle surface. 2) A significant positive correlation was measured between the amount of apoAI and the CE bound by _{IS}HDL NP following incubation with serum. Thus, the potential for CE on the _{IS}HDL NP as a biosensor for detecting variations in serum apoAI has been demonstrated. 3) Data show that CE formed by and sequestered within _{IS}HDL NP is significantly correlated to the total cholesterol efflux to serum measured using the CEA.

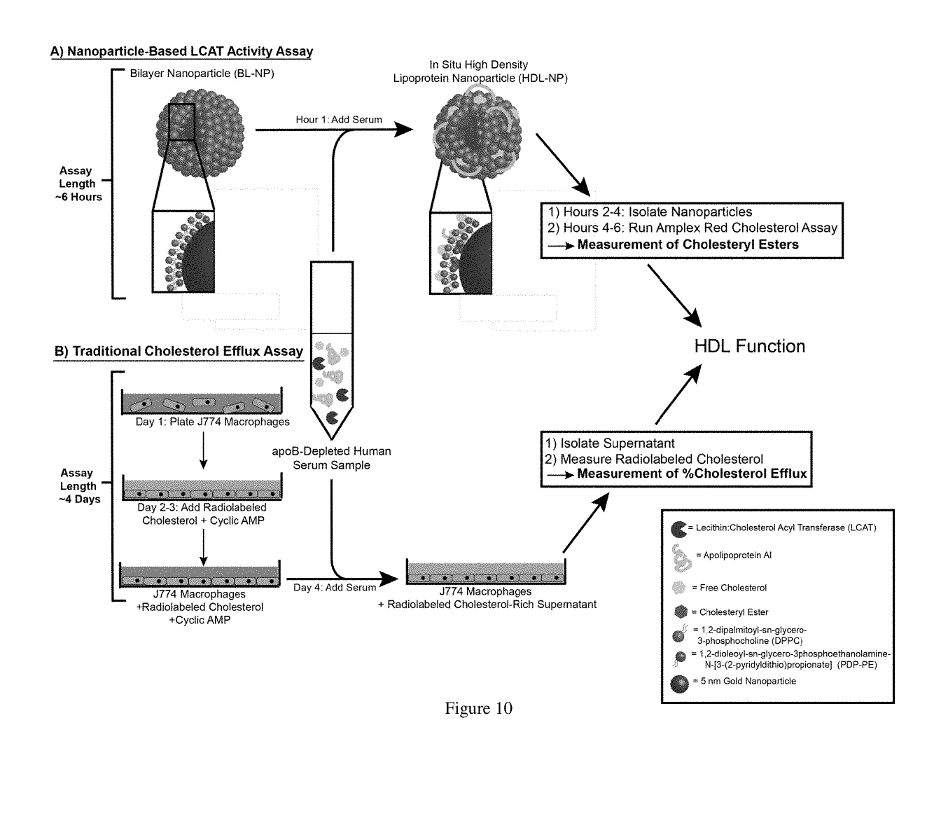

The BL-NP biosensor platform demonstrated in this work provides the potential for a simple, cheap, cell-free assay that, in its current iteration, provides results in a matter of hours and without the use of radioactive materials (FIG. 10). Also, this platform for directly measuring CE sets the foundation for a biosensor sensitive to changes in apoAI and LCAT activity that may result from factors such as therapeutic intervention, dietary modification, or exercise. As above, future work requires correlation with well-annotated and characterized serum samples obtained in a controlled setting from individual patients. Finally, the development of a robust cholesterol oxidase-based sensing device for shortening the proposed assay time and providing faster results for a future point-of-care system is underway.

### REFERENCES

1. Wilson, P. W., R. D. Abbott, and W. P. Castelli. 1988. High density lipoprotein cholesterol and mortality. The Framingham Heart Study. Arteriosclerosis, Thrombosis, and Vascular Biology. 8: 737-741.
2. Chyu, K.-Y., and P. K. Shah. 2015. HDL/ApoA-1 infusion and ApoA-1 gene therapy in atherosclerosis. Front. Pharmacol. 6: 1935.
3. Jonas, A. 1991. Lecithin-cholesterol acyltransferase in the metabolism of high-density lipoproteins. Biochimica et Biophysica Acta (BBA)—Lipids and Lipid Metabolism. 1084: 205-220.
4. Dinnes, D. L. M., S. J. Nicholls, W. Jessup, and L. Kritharides. 2015. HDL heterogeneity and serum efflux capacity. Current Opinion in Lipidology. 26: 350-352.
5. Rothblat, G. H., L. Y. Arbogast, and E. K. Ray. 1978. Stimulation of esterified cholesterol accumulation in tissue culture cells exposed to high density lipoproteins enriched in free cholesterol. The Journal of Lipid Research. 19: 350-358.
6. Khera, A. V., M. Cuchel, M. de la Llera-Moya, A. Rodrigues, M. F. Burke, K. Jafri, B. C. French, J. A. Phillips, M. L. Mucksavage, R. L. Wilensky, E. R. Mohler, G. H. Rothblat, and D. J. Rader. 2011. Cholesterol Efflux Capacity, High-Density Lipoprotein Function, and Atherosclerosis. N. Engl. J. Med. 364: 127-135.
7. Rohatgi, A., A. Khera, J. D. Berry, E. G. Givens, C. R. Ayers, K. E. Wedin, I. J. Neeland, I. S. Yuhanna, D. R. Rader, J. A. de Lemos, and P. W. Shaul. 2014. HDL cholesterol efflux capacity and incident cardiovascular events. N. Engl. J. Med. 371: 2383-2393.
8. la Llera Moya, de, M., V. Atger, J. L. Paul, N. Fournier, N. Moatti, P. Giral, K. E. Friday, and G. Rothblat. 1994. A cell culture system for screening human serum for ability to promote cellular cholesterol efflux. Relations between serum components and efflux, esterification, and transfer. Arterioscler. Thromb. 14: 1056-1065.
9. Sankaranarayanan, S., G. Kellner-Weibel, M. de la Llera-Moya, M. C. Phillips, B. F. Asztalos, R. Bittman, and G. H. Rothblat. 2011. A sensitive assay for ABCA1-mediated cholesterol efflux using BODIPY-cholesterol. J. Lipid Res. 52: 2332-2340.
10. Phillips, M. C. 2014. Molecular mechanisms of cellular cholesterol efflux. J. Biol. Chem. 289: 24020-24029.
11. Wang, M., H. Guo, S. Wang, R. Yang, H. Li, H. Zhao, S. Wang, J. Dong, and W. Chen. 2014. The measurement of high-density lipoprotein mediated cholesterol efflux from macrophage cells by liquid chromatography tandem mass spectrometry. Cell. Physiol. Biochem. 34: 1901-1911.
12. Remaley, A. T. 2015. HDL cholesterol/HDL particle ratio: a new measure of HDL function? J. Am. Coll. Cardiol. 65: 364-366.
13. Cormode, D. P., K. C. Briley-Saebo, W. J. M. Mulder, J. G. S. Aguinaldo, A. Barazza, Y. Ma, E. A. Fisher, and Z. A. Fayad. 2008. An ApoA-I Mimetic Peptide High-Density-Lipoprotein-Based MRI Contrast Agent for Atherosclerotic Plaque Composition Detection. Small. 4: 1437-1444.
14. Chen, W., D. P. Cormode, Y. Vengrenyuk, B. Herranz, J. E. Feig, A. Klink, W. J. M. Mulder, E. A. Fisher, and Z. A. Fayad. 2013. Collagen-specific peptide conjugated HDL nanoparticles as MRI contrast agent to evaluate compositional changes in atherosclerotic plaque regression. JACC Cardiovasc Imaging. 6: 373-384.
15. Duivenvoorden, R., J. Tang, D. P. Cormode, A. J. Mieszawska, D. Izquierdo-Garcia, C. Ozcan, M. J. Otten, N. Zaidi, M. E. Lobatto, S. M. van Rijs, B. Priem, E. L. Kuan, C. Martel, B. Hewing, H. Sager, M. Nahrendorf, G. J. Randolph, E. S. G. Stroes, V. Fuster, E. A. Fisher, Z. A. Fayad, and W. J. M. Mulder. 2014. A statin-loaded reconstituted high-density lipoprotein nanoparticle inhibits atherosclerotic plaque inflammation. Nat Commun. 5: 3065.
16. Thaxton, C. S., W. L. Daniel, D. A. Giljohann, A. D. Thomas, and C. A. Mirkin. 2009. Templated Spherical High Density Lipoprotein Nanoparticles. J. Am. Chem. Soc. 131: 1384-1385.
17. Sun, W., W. Wu, K. M. McMahon, J. S. Rink, and C. S. Thaxton. 2016. Mosaic Interdigitated Structure in Nanoparticle-Templated Phospholipid Bilayer Supports Partial Lipidation of Apolipoprotein A-I. Part Part Syst Charact. 33: 300-305.
18. Lai, C.-T., W. Sun, R. U. Palekar, C. S. Thaxton, and G. C. Schatz. 2017. Molecular Dynamics Simulation and Experimental Studies of Gold Nanoparticle Templated HDL-like Nanoparticles for Cholesterol Metabolism Therapeutics. ACS Appl Mater Interfaces. 9: 1247-1254.
19. Luthi, A. J., N. N. Lyssenko, D. Quach, K. M. McMahon, J. S. Millar, K. C. Vickers, D. J. Rader, M. C. Phillips, C. A. Mirkin, and C. S. Thaxton. 2015. Robust passive and active efflux of cellular cholesterol to a designer functional mimic of high density lipoprotein. The Journal of Lipid Research. 56: 972-985.
20. Lai, C.-T., W. Sun, R. U. Palekar, C. S. Thaxton, and G. C. Schatz. 2017. Molecular Dynamics Simulation and Experimental Studies of Gold Nanoparticle Templated HDL-like Nanoparticles for Cholesterol Metabolism Therapeutics. ACS Appl Mater Interfaces. 9: 1247-1254.
21. Luthi, A. J., H. Zhang, D. Kim, D. A. Giljohann, C. A. Mirkin, and C. S. Thaxton. 2012. Tailoring of Biomimetic High-Density Lipoprotein Nanostructures Changes Cholesterol Binding and Efflux. ACS Nano. 6: 276-285.
22. Cui, L., Y. Li, M. Lu, B. Tang, and C.-Y. Zhang. 2017. An ultrasensitive electrochemical biosensor for polynucleotide kinase assay based on gold nanoparticle-mediated lambda exonuclease cleavage-induced signal amplification. Biosens Bioelectron. 99: 1-7.
23. Sun, K., Y. Chang, B. Zhou, X. Wang, and L. Liu. 2017. Gold nanoparticles-based electrochemical method for the detection of protein kinase with a peptide-like inhibitor as the bioreceptor. International Journal of Nanomedicine. 12: 1905-1915.
24. Amini, B., M. Kamali, M. Salouti, and P. Yaghmaei. 2017. Fluorescence bio-barcode DNA assay based on gold and magnetic nanoparticles for detection of Exotoxin A gene sequence. Biosens Bioelectron. 92: 679-686.
25. Kurzqtkowska, K., T. Santiago, and M. Hepel. 2017. Plasmonic nanocarrier grid-enhanced Raman sensor for studies of anticancer drug delivery. Biosens Bioelectron. 91: 780-787.
26. Cao, Y. C., R. Jin, J.-M. Nam, C. S. Thaxton, and C. A. Mirkin. 2003. Raman Dye-Labeled Nanoparticle Probes for Proteins. J. Am. Chem. Soc. 125: 14676-14677.
27. Thaxton, C. S., H. D. Hill, D. G. Georganopoulou, S. I. Stoeva, and C. A. Mirkin. 2005. A Bio-Bar-Code Assay Based upon Dithiothreitol-Induced Oligonucleotide Release. Anal. Chem. 77: 8174-8178.
28. Thaxton, C. S., R. Elghanian, A. D. Thomas, S. I. Stoeva, J.-S. Lee, N. D. Smith, A. J. Schaeffer, H. Klocker, W. Horninger, G. Bartsch, and C. A. Mirkin. 2009. Nanoparticle-based bio-barcode assay redefines “undetectable” PSA and biochemical recurrence after radical prostatectomy. Proceedings of the National Academy of Sciences. 106: 18437-18442.
29. Taton, T. A., C. A. Mirkin, and R. L. Letsinger. 2000. Scanometric DNA Array Detection with Nanoparticle Probes. Science. 289: 1757-1760.
30. Elghanian, R., J. J. Storhoff, R. C. Mucic, R. L. Letsinger, and C. A. Mirkin. 1997. Selective colorimetric detection of polynucleotides based on the distance-dependent optical properties of gold nanoparticles. Science. 277: 1078-1081.
31. Rosenson, R. S., H. B. Brewer, W. S. Davidson, Z. A. Fayad, V. Fuster, J. Goldstein, M. Hellerstein, X.-C. Jiang, M. C. Phillips, D. J. Rader, A. T. Remaley, G. H. Rothblat, A. R. Tall, and L. Yvan-Charvet. 2012. Cholesterol efflux and atheroprotection: advancing the concept of reverse cholesterol transport. Circulation. 125: 1905-1919.
32. Sun, W., W. Wu, K. M. McMahon, J. S. Rink, and C. S. Thaxton. 2016. Mosaic Interdigitated Structure in Nanoparticle-Templated Phospholipid Bilayer Supports Partial Lipidation of Apolipoprotein A-I. Part Part Syst Charact. 33: 300-305.
33. Mandy Ali, K., A. Wonnerth, K. Huber, and J. Wojta. 2012. Cardiovascular disease risk reduction by raising HDL cholesterol—current therapies and future opportunities. British Journal of Pharmacology. 167: 1177-1194.
34. Hutchins, P. M., and J. W. Heinecke. 2015. Cholesterol efflux capacity, macrophage reverse cholesterol transport and cardioprotective HDL. Current Opinion in Lipidology. 26: 388-393.
35. Saleheen, D., R. Scott, S. Javad, W. Zhao, A. Rodrigues, A. Picataggi, D. Lukmanova, M. L. Mucksavage, R. Luben, J. Billheimer, J. J. P. Kastelein, S. M. Boekholdt, K.-T. Khaw, N. Wareham, and D. J. Rader. 2015. Association of HDL cholesterol efflux capacity with incident coronary heart disease events: a prospective case-control study. Lancet Diabetes Endocrinol. 3: 507-513.
36. Frank, P. G., and Y. L. Marcel. 2000. Apolipoprotein A-I: structure-function relationships. The Journal of Lipid Research. 41: 853-872.
37. Gu, X., Z. Wu, Y. Huang, M. A. Wagner, C. Baleanu-Gogonea, R. A. Mehl, J. A. Buffa, A. J. DiDonato, L. B. Hazen, P. L. Fox, V. Gogonea, J. S. Parks, J. A. DiDonato, and S. L. Hazen. 2016. A Systematic Investigation of Structure/Function Requirements for the Apolipoprotein A-I/Lecithin Cholesterol Acyltransferase Interaction Loop of High-density Lipoprotein. Journal of Biological Chemistry. 291: 6386-6395.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used.

Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures.

1. A device comprising:
a housing,
a blood extraction element connected at least in part, either directly or indirectly to an external portion of the housing,
a nanostructure comprising a core and a phospholipid shell capable of binding to a lecithin:cholesterol acyltransferase (LCAT) activator within at least a portion of the housing.

2. The device of claim 1, wherein the device is a wearable or portable device.

3. The device of claim 1, wherein the nanostructure comprises a solid core.

4. The device of claim 1, wherein the nanostructure comprises a solid core and a lipid layer.

5. The device of claim 1, wherein the nanostructure binds the LCAT activator.

6. The device of claim 1, wherein the LCAT activator is an apolipoprotein and wherein the LCAT activator may be detected directly.

7. The device of claim 6, wherein the nanostructure has a gold core and a lipid bilayer or monolayer.

8.-17. (canceled)

18. A composition comprising a lipid functionalized nanoparticle having an inorganic core and a phospholipid shell, wherein the lipid functionalized nanoparticle comprises a fluorescently labeled lipid, including phospholipid, cholesterol lipid, and non-phosphorous containing di-acyl lipid.

19.-50. (canceled)

51. A method for synthesizing a nanostructure in situ, the method comprising incubating a nanostructure comprising a nanostructure core comprising an inorganic material, a shell comprising a lipid layer, surrounding and attached to the nanostructure core, the shell having an inner surface and an outer surface, with a biological sample for a time sufficient to sequester one or more apolipoproteins from the biological sample.

52. The method of claim 51, wherein the lipid layer is a lipid bilayer.

53. (canceled)

54. A method for synthesizing a nanostructure in situ, the method comprising incubating a nanostructure comprising an inorganic core, a lipid shell, surrounding and attached to the inorganic core, the shell having an inner surface and/or an outer surface, with a biological sample for a time sufficient to sequester one or more apolipoproteins present in the biological sample.

55. (canceled)

56. The method of claim 54, further comprising administering the biological sample to a subject as a therapeutic.

57. A method for sequestering cholesterol in a subject, comprising
administering to a subject a nanostructure consisting essentially of an inorganic core, a lipid shell, surrounding and attached to the inorganic core, the shell having an inner surface and/or an outer surface, wherein the nanostructure is capable of sequestering apolipoprotein in vivo, which sequesters cholesterol.

58. The method of claim 57, wherein the lipid shell is comprised of phospholipids.

59. The method of claim 57, wherein the subject has a disease associated with high cholesterol, and optionally, wherein the disease associated with high cholesterol is selected from the group consisting of cardiovascular disease, atherosclerosis, hyperlipidemia, cancer, inflammation, a protein storage disease, a disease of hemostasis, a rheumatic disease, or a neurologic disease.

60. (canceled)

61. A therapeutic or diagnostic composition, comprising:
a nanostructure consisting essentially of an inorganic core and a lipid shell, surrounding and attached to the inorganic core, wherein the nanoparticle is formulated in a pharmaceutically acceptable carrier.

62. The composition of claim 61, wherein the lipid shell is a lipid bilayer or lipid monolayer.

63. (canceled)

64. The composition of claim 61, wherein the lipids in the shell are comprised of 1,2-dipalmitoyl-sn-glycero-3-phosphothioethanol (DPPTE), phosphotidylcholine (PC) and 1,2-dipalmitoyl-sn-glycero-3-phosphocholine (DPPC).

65. The composition of claim 61, wherein the core is a gold core.

66. The composition of claim 65, wherein the gold core is 5-6 nm in diameter.

67. The composition of claim 61, wherein the composition further comprises an apolipoprotein, and optionally, wherein the apolipoprotein is apolipoprotein AI (Apo-AI).

68. The composition of claim 61, wherein the nanostructure is constructed and arranged to sequester 2-4 Apo-AI molecules.

69. The composition of claim 61, wherein there are 71-95 lipids in the shell.

70. The composition of claim 61, wherein the lipids are phospholipids.
